(19) United States
(12) Patent Application Publication
(10) Pub. No.: US 2026/0297525 A1
(43) Pub. Date: Oct. 1, 2026

(54) PATROLLING MONOCYTE CELLULAR THERAPY

(71) Applicant: New York Blood Center, Inc., Rye, NY (US)

(72) Inventors: Yunfeng Liu, New York, NY (US); Karina Yazdanbakhsh, New York, NY (US)

(21) Appl. No.: 19/476,994

(22) PCT Filed: Apr. 22, 2024
(86) PCT No.: PCT/US2024/025676

§ 371 Date: Oct. 20, 2025

Related U.S. Application Data
(60) Provisional application No. 63/497,638, filed on Apr. 21, 2023.
(60) Provisional application No. 63/517,710, filed on Aug. 4, 2023.
(60) Provisional application No. 63/582,169, filed on Sep. 12, 2023.

(51) Int. Cl. C12N 5/0786; A61K 35/15; A61K 38/19; A61K 40/17; A61K 40/50; A61P 7/02; C07K 16/28; C12N 5/00; G01N 33/50; G01N 33/68

(52) U.S. Cl. CPC C12N 5/0645; A61K 35/15; A61K 38/193; A61K 40/17; A61K 40/50; A61P 7/02; C07K 16/2854; C12N 5/0087; G01N 33/5044; G01N 33/5094; G01N 33/6863; G01N 33/6893; C12N 2501/22; C12N 2501/58; C12N 2502/1323; C12N 2502/1352; C12N 2502/28; C12N 2521/00; G01N 2333/53; G01N 2800/22; G01N 2800/50; G01N 2800/52

(57) ABSTRACT

Disclosed herein are methods of expanding populations of patrolling monocytes in vitro and in vivo for treatment of sickle-cell disease (SCD), reducing pain and vaso-occlusive events in SCD patients, promoting wound healing, reducing tumor metastases, and treating vascular inflammatory conditions in subjects in need thereof.

### CROSS-REFERENCE TO RELATED APPLICATIONS

The present application claims the benefit of U.S. Provisional applications 63/497,638 filed Apr. 21, 2023, 63/517,710 filed Aug. 7, 2023, and 63/582,169 filed Sep. 12, 2023, the entire contents of all of which are incorporated by reference herein.

### STATEMENT REGARDING FEDERALLY-SPONSORED RESEARCH

This invention was made in part with government support under Grant No. R35HL 161239 awarded by the National Institutes of Health. The government has certain rights in the invention.

### BACKGROUND

Sickle cell disease (SCD) is characterized by hemolytic anemia and painful vaso-occlusive events (VOE, also known as vaso-occlusive crises (VOC)), caused by increased adherence of sickle red blood cells (RBCs) to the underlying activated vascular endothelium. Accumulating evidence highlights the pivotal role of the mononuclear phagocyte system, encompassing blood monocytes, spleen red pulp macrophages, and liver Kupffer cells in erythrophagocytosis of sickle RBC and in the clearance of hemolytic by-products in SCD. A subset of blood monocytes, known as non-classical monocytes or patrolling monocytes (PMo), are instrumental in mitigating VOCs in SCD by scavenging endothelial cell (EC)-attached sickle RBCs and debris from hemolysis-damaged endothelium (Liu et al. Blood 131:1600-1610, 2018); Liu et al. Blood 134:579-590, 2019; Liu et al. Transfus Clin Biol 26:128-129, 2019). In comparison to healthy controls, SCD patients exhibit reduced circulating PMo levels. During sickle crises, PMo frequency further declines as a consequence of increased erythrophagocytosis of EC-bound sickle erythrocytes, overwhelming the compensatory PMo survival mechanisms, and resulting in PMo death.

PMo primarily differentiate from classical monocytes (CMo) in the bone marrow (BM) and circulation. Under normal conditions, CMo demonstrate a short circulatory lifespan, with the majority (90-99%) transmigrating across the vascular endothelium into tissues, where they differentiate into tissue macrophages or dendritic cells. In contrast, a small proportion (1-10%) of CMo follow an alternative differentiation pathway, becoming blood PMo which, relative to CMo, have an extended lifespan and limited transmigration into tissues.

In the context of SCD, despite lower frequency of PMo, CMo numbers and levels of the CMo chemokine (chemokine (C—C motif) ligand 2 (CCL2)) which promotes CMo migration to tissues, are increased. A recent study demonstrated an expansion of monocyte-derived macrophages in the liver and to a lesser extent in spleen due to intravascular hemolysis (Liu et al. Blood 138:1162-1171, 2021).

### SUMMARY

Disclosed herein are methods of differentiating monocytes into patrolling monocytes ex vivo, the method comprising: obtaining classical monocytes; and culturing the classical monocytes in the presence of colony-stimulating factor-1 (CSF-1); wherein as a result of the culture conditions, the classical monocytes differentiate into patrolling monocytes. In some embodiments, the patrolling monocytes have the phenotype CD43⁺CD163⁻.

In some embodiments, the classical monocytes are isolated from a subject. In some embodiments, the classical monocytes are purified from the bone marrow monocytes. In some embodiments, the classical monocytes are derived from induced pluripotent stem cells (iPSC). In some embodiments, the classical monocytes are derived from HLA homozygous iPSC.

In some embodiments, the classical monocytes are cultured in the presence of feeder cells. In some embodiments, the feeder cells are endothelial cells, mesenchymal stromal cells, or fibroblasts. In some embodiments, the feeder cells are pre-treated with one or more of an antibody specific for P-selectin, and antibody specific for vCAM-1, an antibody specific for CD11b, or an antibody to ICAM-1 prior to culturing with the classical monocytes. In some embodiments, the culture takes place in a transwell culture system.

In some embodiments, the classical monocytes are cultured in the absence of feeder cells. In some embodiments, the classical monocytes are cultured in cell repellent tissue culture dishes.

In some embodiments, the classical monocytes are cultured in the presence of a shear stress.

Also disclosed herein are methods of expanding the number of patrolling monocytes in a subject, the method comprising: administering to the subject CSF-1 and an antibody specific for P-selectin; wherein the method induces the differentiation of classical monocytes to patrolling monocytes in the subject and thus increases the number of patrolling monocytes in the subject.

Disclosed herein are methods of increasing the number of patrolling monocytes in a subject comprising increasing the levels of CSF-1 and/or decreasing the levels of CCl2 in the subject.

Disclosed herein are methods of reducing vaso-occlusive events and/or pain in a subject with sickle-cell disease (SCD) comprising increasing CSF-1 and/or decreasing CCl2 in the subject.

Also disclosed herein are methods of treating sickle cell disease (SCD) in a subject in need thereof comprising: administering to the subject CSF-1 and an antibody specific for P-selectin; wherein the method increases the number of patrolling monocytes in the subject, thereby treating the SCD.

Also disclosed herein is the use of patrolling monocytes in the treatment of SCD in a subject in need thereof comprising: administering to the subject CSF-1 and an antibody specific for P-selectin; wherein as a result of the administration, the number of patrolling monocytes is increased in the subject, thereby treating the SCD. In some embodiments, the combination of CSF-1 and an antibody specific for P-selectin treats a vaso-occlusive event in the subject better than the antibody specific for P-selectin alone. In some embodiments, the antibody specific for P-selectin is crizanlizumab.

Disclosed herein are methods of treating SCD in a subject in need thereof, comprising administering to the subject ex vivo produced patrolling monocytes. Disclosed herein is the use of ex vivo produced patrolling monocytes for the treatment of SCD in a subject in need thereof.

Disclosed herein are methods of treating a vascular inflammatory condition in a subject in need thereof, comprising administering to the subject ex vivo produced patrolling monocytes. Disclosed herein is the use of ex vivo produced patrolling monocytes for the treatment of a vascular inflammatory condition in a subject in need thereof.

Disclosed herein are methods of promoting wound healing in a subject in need thereof, comprising administering to the subject ex vivo produced patrolling monocytes. Disclosed herein is the use of ex vivo produced patrolling monocytes for promoting wound healing in a subject in need thereof.

Disclosed herein are methods of reducing tumor metastasis in a subject in need thereof, comprising administering to the subject ex vivo produced patrolling monocytes. Disclosed herein is the use of ex vivo produced patrolling monocytes for reducing tumor metastasis in a subject in need thereof.

In some embodiments, the patrolling monocytes are produced by the method disclosed herein. In some embodiments, the patrolling monocytes are autologous to the subject. In some embodiments, the patrolling monocytes are allogenic to the subject. In some embodiments, the allogeneic patrolling monocytes are HLA matched to the subject.

Also disclosed herein are methods of monitoring the efficacy of a pharmaceutical agent in the treatment of SCD or a vaso-occlusive events in SCD in a subject in need thereof, the method comprising: (a) measuring the ratio of CSF-1 to CCL2 (CSF-1/CCL2) in the blood of the subject, wherein the CSF-1/CCL2 ratio is indicative of the efficacy or expected efficacy of the pharmaceutical agent in treating SCD; or (b) measuring the number of blood patrolling monocytes in the subject, wherein a high level of blood patrolling monocytes is indicative of the efficacy or expected efficacy of the pharmaceutical agent in treating SCD.

In some embodiments, the pharmaceutical agent is an agent effective for treatment of SCD or effective for the prevention of vaso-occlusive events in SCD. In some embodiments, the pharmaceutical agent is L-glutamine, voxelotor, hydroxyurea, or an antibody specific for P-selectin. In some embodiments, the antibody specific for P-selectin is crizanlizumab.

In some embodiments, a high ratio of CSF-1/CCL2 is predictive, or indicative, of efficacy of the antibody in treatment of SCD or a vaso-occlusive events in SCD. In some embodiments, a low ratio of CSF-1/CCL2 is predictive, or indicative, of non-efficacy of the antibody in treatment of SCD or a vaso-occlusive events in SCD. In some embodiments, a high number of patrolling monocytes is predictive, or indicative, of efficacy of the antibody in treatment of SCD or a vaso-occlusive event in SCD. In some embodiments, a low number of patrolling monocytes. is predictive, or indicative, of non-efficacy of the antibody in treatment of SCD or a vaso-occlusive event in SCD. In some embodiments, the ratio of CSF-1/CCL2 or the number of patrolling monocytes are measured before initiation of antibody therapy. In some embodiments, the ratio of CSF-1/CCL2 or the number of patrolling monocytes are measured after initiation of antibody therapy. In some embodiments, the ratio of CSF-1/CCL2 or the number of patrolling monocytes are measured more than six months after the initiation of antibody therapy. In some embodiments, the determination of the ratio of CSF-1/CCL2 or the number of patrolling monocytes causes a change in the therapy for the subject. In some embodiments, efficacy is a reduction in the number of vaso-occlusive events in the subject.

Also disclosed herein are methods of determining the risk of vaso-occlusive events in SCD in a subject in need thereof, the method comprising (a) measuring the number of blood patrolling monocytes in the subject, wherein a low level of blood patrolling monocytes is a risk factor for a vaso-occlusive event; or (b) measuring the ratio of CSF-1 to CCL2 (CSF-1/CCL2) in the blood of the subject, wherein a lower CSF-1/CCL2 ratio is a risk factor for a vaso-occlusive event.

### BRIEF DESCRIPTION OF THE DRAWINGS

FIG. 1A-H depict the assessment of plasma CSF-1 levels and induction in SCD. (FIG. 1A) Plasma CSF-1 levels in HD (n=13) and SCD patients at steady state (n=30). (FIG. 1B) Plasma CSF-1 levels in control and sickle mice (n=5). (FIG. 1C) The absolute number of circulating Ly-6C^{hi} CMo and Ly-6C^{lo/-} PMo in sickle mice (n=3) on day 3 and 5 post s.c. injection with CSF-1 (0.5 mg/kg body weight/day) or PBS (200 μl/20 g body weight/day). (FIG. 1D) The absolute number of circulating Ly-6C^{hi} CMo and Ly-6C^{lo/-} PMo in sickle mice (n=4-5) at 72 hr following i.p. injection with anti-CSF-1 blocking antibody (1 mg/kg body weight) or isotype antibody (1 mg/kg body weight). (FIG. 1E) Plasma CSF-1 levels in WT mice at 20 hr after i.v. injection of PBS as control (200 μl/20 g body weight), RBC lysate (17.5 μmol hemoglobin/kg body weight), or hemin (17.5 μmol/kg body weight) (n=4-7). (FIG. 1F) Plasma CSF-1 levels in WT mice at 20 hr after i.v. injection with hemin at doses of 0, 8.8, 17.5, or 35 μmol/kg body weight (n=3-9). (FIG. 1G) Plasma CSF-1 levels in WT mice at time points of 0, 6, 20, and 72 hr post i.v. injection with hemin (35 μmol/kg body weight) (n=4-6). (FIG. 1H) Plasma CSF-1 levels in WT mice 20 hours after i.v. injection with hemin combined with PBS (17.5 μmol/kg body weight), hemin and hemopexin (17.5 μmol/kg body weight), or PBS (200 μl/20 g body weight) as control (n=4-7). Data are represented as mean±SEM and compared using a two-tailed Student's t-test. * p<0.05.

FIG. 2A-F depict the mechanism of CSF-1 induction by hemolysis. (FIG. 2A) Plasma CSF-1 levels in WT mice, TLR4^{-/-} mice and Ifnar1^{-/-} mice at 20 hr post i.v. injection with hemin (35 μmol/kg body weight) (n=3-4). (FIG. 2B) Plasma CSF-1 levels in Vavi1^{cre}Nrf2^{+/+} mice and control Nrf2^{+/+} mice at 20 hr post i.v. injection with hemin (35 μmol/kg body weight) (n=3-5). (FIG. 2C) Representative histogram and (FIG. 2D) bar graph showing CSF-1 expression in liver EC from control mice (dashed line, n=3) and sickle mice (solid line, n=3). Isotype control is shown as gray-filled histogram. (FIG. 2E) Representative histogram and (FIG. 2F) bar graph showing CSF-1 expression in liver EC from control mice (dashed line, n=3) and hemin-treated mice (solid line, n=3). Gray-filled histogram represents isotype control. Data are represented as mean±SEM and compared using a two-tailed Student's t-test. * p<0.05.

FIG. 3A-H depict the relationship between PMo numbers and the ratio of CSF-1/CCL-2. (FIG. 3A) Scatter plot analysis showing correlation relationship between plasma CSF-1 levels and absolute numbers of circulating PMo in patients with SCD (n=30, image includes human PMo gating strategy). (FIG. 3B) Scatter plot analysis showing correlation relationship between the ratio of plasma CSF-1 vs CCL-2 levels and absolute numbers of circulating PMo in patients with SCD (n=30). (FIG. 3C) Plasma CSF-1 levels and (FIG. 3D) plasma CCL-2 levels in C57BL/6 mice and FVB mice 20 hr after i.v. injection with hemin (17.5 μmol/kg body weight), or PBS (200 μl/20 g body weight) as control (n=5-9). Absolute number of circulating Ly-6C^{hi} CMo (FIG. 3E) and Ly-6C^{lo/-} PMo (FIG. 3F) in mice at time point of 3 days after injection as shown in FIG. 3C (n=6). Absolute number of liver Ly-6C^{hi}MHC-II⁻ CMo (FIG. 3G) and Ly-6C⁺MHC-II⁺transient macrophage (tMφ) (FIG. 3H) in mice injected as in FIG. 3C (n=5-9). The correlation analysis in FIG. 3A and FIG. 3B was determined by Spearman Rho. Symbols represent data from individual mice. Data are represented as mean±SEM and compared using a two-tailed Student's t-test. * p<0.05.

FIG. 4A-F depict CMo migration blockade promotes differentiation into PMo in vitro and in vivo. (FIG. 4A) Schematic representation of experimental design. Transwell culture of purified BM Ly-6C^{hi}MHC-II⁻ CMo placed above mouse endothelial cells (ECs) seeded in the upper compartment for 2 days. Monocytes in the bottom compartment are considered as having trans-migrated through the ECs while the ones remaining in the top well are the non-migrated subpopulation. (FIG. 4B) Representative histograms showing the gating strategy for Ly-6C^{hi}MHC-II⁻ CMo, Ly-6C^{lo/-}MHC-II⁻ PMo, and Ly-6C⁺MHC-II⁺transient macrophage (tMφ) in the transwell culture as shown in FIG. 4A. (FIG. 4C) Bar graph showing expression of surface markers on cultured monocytes as shown in FIG. 4A (n=6). (FIG. 4D) Bar graph showing the monocyte numbers in co-cultures of purified BM Ly-6C^{hi}MHC-II⁻ CMo layered above mouse ECs which had been pre-treated with blocking antibody against P-selectin, VCAM-1, ICAM-1, E-selectin, CD11b, or isotype control (10 ng/ml, n=6). (FIG. 4E) Bar graph showing absolute number of circulating Ly-6C^{hi} CMo and Ly-6C^{lo/-} PMo at 20 hr in hemin (35 μmol/kg body weight)−injected WT mice pretreated for 30 min with anti-P-selectin blocking antibody (5 mg/kg body weight, i.v.), anti-VCAM-1 blocking antibody (5 mg/kg body weight, i.v), or isotype control antibody (5 mg/kg body weight, i.v) (n=6). (FIG. 4F) Bar graph showing absolute number of liver Ly-6C^{hi}MHC-II⁻ CMo and Ly-6C⁺MHC-II⁺transient macrophage (tMφ) in mice injected as in FIG. 4D (n=6). Symbols represent data from individual mice. Data are represented as mean±SEM and compared using two-way ANOVA with Bonferroni's multiple comparisons. * p<0.05.

FIG. 5A-H depicts the effects of anti-P-selectin blockade and CSF-1 treatment on monocyte subpopulations and liver pathology in sickle mice. Bar graph showing the absolute numbers of circulating Ly-6C^{hi} CMo (FIG. 5A) and Ly-6C^{lo/-} PMo (FIG. 5B) in sickle mice at day 5 post-administration with anti-P-selectin blocking antibody or isotype control antibody alone (5 mg/kg body weight, i.p. every other day) or with s.c. injection with CSF-1 (0.5 mg/kg body weight/day) (n=5). (FIG. 5C) Representative hematoxylin and eosin-stained liver sections from mice injected as in FIG. 5A (scale=100 μm). Black arrows indicate RBC stasis within blood vessels. (FIG. 5D) The frequency of occluded blood vessels (% stasis) in liver sections from mice injected as in FIG. 5A (n=6-8). Bar graph showing the absolute number of liver Ly-6C^{hi}MHC-II⁻ CMo (FIG. 5E) and Ly-6C⁺MHC-II⁺transient macrophage (tMφ) (FIG. 5F) in mice injected as in FIG. 5A (n=5). (FIG. 5G) Representative TUNEL-stained liver sections from mice injected as in FIG. 5A (scale=100 μm). Black arrows indicate TUNEL positive area. (FIG. 5H) The frequencies of necrotic areas (TUNEL positive) in liver sections from mice injected as in FIG. 5A (n=6-8). Symbols represent data from individual mice. Data are represented as mean±SEM and compared using a two-tailed Student's t-test. * p<0.05.

FIG. 6A-G depict monocyte subpopulations in mouse blood and response to CSF-1 and MDP treatments. (FIG. 6A) Gating strategy for mouse blood classical monocytes (CMo, CD11b⁺CD115⁺Ly-6C^{hi}) and patrolling monocytes (PMo, CD11b⁺CD115⁺Ly-6C^{lo/-}) in single live CD45+ leukocytes. (FIG. 6B) Absolute number of circulating Ly-6C^{hi} CMo and Ly-6C^{lo/-} PMo in WT mice (n=6) at day 3 and 5 post s.c. injection with CSF-1 (0.5 mg/kg body weight/day) or PBS control (200 μl/20 g body weight/day). (FIG. 6C) Blood neutrophil numbers, (FIG. 6D) Blood RBC numbers, and (FIG. 6E) blood hemoglobin (Hgb) levels in sickle mice at day 5 post s.c. injection with CSF-1 (0.5 mg/kg body weight/day) as in FIG. 1C (n=5-6). (FIG. 6F) Plasma CSF-1 levels in WT mice at 20 hr after i.v. injection with MDP (1 mg/kg body weight) or PBS as control (n=6-8). (FIG. 6G) Absolute number of circulating Ly-6C^{hi} CMo and Ly-6C^{lo/-} PMo in WT mice at day 5 post treatment with MDP (1 mg/kg body weight/day) and blocking antibody against CSF-1 or isotype control (1 mg/kg body weight/two days) (n=6). Symbols represent data from individual mice. Data are represented as mean±SEM and compared using a two-tailed Student's t-test in all the Figures except FIGS. 6B and 6G which were compared using two-way ANOVA with Bonferroni's multiple comparisons. * p<0.05.

FIG. 7A-D depict mouse liver endothelial cell and leukocyte subpopulation gating strategy and CSF-1 expression. (FIG. 7A) Gating strategy for mouse liver endothelial cells (EC) as single ViViD⁻CD45⁻CD31⁺ cells, leukocytes as single ViViD⁻CD45⁺ cells, and leukocyte subsets as resident Mφ: F4/80^{hi}CD11b^{lo}CD64⁺Tim-4⁺CD11c^{lo/-}CD24^{lo/-} leukocytes, CMo: Ly-6C^{hi}MHC-II⁻ CD11b^{lo}CD64⁺CD11c^{lo/-}CD24^{lo/-} leukocytes, Ly-6C⁺MHC-II⁺transient macrophage (tMφ): Ly-6C⁺MHC-II⁺CD11b/CD64⁺CD11c^{lo/-}CD24^{lo/-} leukocytes. (FIG. 7B) scatter plot with bar showing CSF-1 expression in liver resident Mφ from WT mice at 20 hr post treatment with PBS as control (200 μl/20 g body weight) or hemin (35 μmol/kg body weight) (n=3). (FIG. 7C) scatter plot with bar showing CSF-1 expression in liver resident Mφ from sickle mice or control mice (n=3). (FIG. 7D) CSF-1 expression in human CMo from SCD patient (n=13) and HD (n=6) (GSE149050). Data are represented as mean±SEM and compared using a two-tailed Student's t-test. * p<0.05.

FIG. 8A-I depict the correlation between plasma CCL-2 levels and circulating PMo numbers, and response to hemin and MDP treatments. (FIG. 8A) Gating strategy for human blood PMo (HLA-DR⁺SSC^{lo}CD14^{lo/-}CD16⁺) in single live CD45⁺PBMCs. (FIG. 8B) Scatter plot analysis showing correlation relationship between plasma CCL-2 levels and absolute numbers of circulating PMo in patients with SCD (n=30 as in FIG. 1). (FIG. 8C) Plasma IFN-α levels in WT C57BL/6 mice and FVB mice 20 hr after i.v. injection with hemin (35 μmol/kg body weight), or PBS (200 μl/20 g body weight) as control (n=6). Plasma CSF-1 levels (FIG. 8D) and CCL-2 levels (FIG. 8E) in sickle mice (n=6-8) one day after treatment with MDP (1 mg/kg body weight) or PBS control. (FIG. 8F) Absolute number of liver Ly-6C^{hi}MHC-II⁻ CMo and Ly-6C⁺MHC-II⁺transient macrophage (Mφ) in sickle mice treated with MDP or PBS (n=6) as shown in FIG. 8D. (FIG. 8G) Blood neutrophil numbers, (FIG. 8H) blood RBC numbers, and (FIG. 81) blood hemoglobin (Hgb) levels in sickle mice treated with MDP as shown in FIG. 8D (n=8). The correlation analysis in FIG. 8B was determined by Spearman Rho. Symbols represent data from individual mice. Symbols represent data from individual mice. Data are represented as mean±SEM, and compared using a two-tailed Student's t-test in all the Figures except FIGS. 8C and 8F which were compared using two-way ANOVA with Bonferroni's multiple comparisons . . . * p<0.05.

FIG. 9A-F depict the response to hemin, CSF-1, and blocking antibodies treatments in mice. (FIG. 9A) Bar graph showing absolute number of circulating Ly-6C^{hi} CMo and Ly-6C^{lo/-} PMo at 20 hr in hemin (35 μmol/kg body weight)-injected WT mice pretreated for 30 min with anti-E-selectin blocking antibody (5 mg/kg body weight, i.v.), or isotype control antibody (5 mg/kg body weight, i.v) (n=5-8). (FIG. 9B) Bar graph showing absolute number of liver Ly-6C^{hi}MHC-II⁻ CMo and Ly-6C⁺MHC-II⁺transient macrophage (tMφ) in mice treatment as in A (n=5-6). Absolute number of liver Ly-6C^{hi}MHC-II⁻ CMo (FIG. 9C) and Ly-6C⁺MHC-II⁺transient macrophage (tMφ) (FIG. 9D) in WT mice and Nr4a1^{-/-} mice 20 hours after i.v. injection with hemin (35 μmol/kg body weight), or PBS (200 μl/20 g body weight) as control (n=5-7). (FIG. 9E) Bar plot showing absolute number of liver resident macrophage (MP) in sickle mice at day 5 post anti-P-selectin blocking antibody or isotype control antibody (5 mg/kg body weight, i.p. every other day), with or without s.c. injection with CSF-1 (0.5 mg/kg body weight/day) as shown in FIG. 5 (n=6). (FIG. 9F) Bar plot showing absolute number of spleen macrophage (Mφ) in sickle mice at day 5 post s.c. injection with CSF-1 (0.5 mg/kg body weight/day) or PBS as control (n=5-6). Symbols represent data from individual mice. Data are represented as mean±SEM, and compared using two-way ANOVA with Bonferroni's multiple comparisons in FIG. 9A-E, and using a two-tailed Student's t-test in FIG. 9F. * p<0.05.

FIG. 10A-B depicts differential marker expression in naive CMos, naïve PMos, and monocyte-derived macrophage (MoMφs). (FIG. 10A) Representative histograms comparing marker expressions in heathy donor (HD) naive circulating CMo (dashed line) and MoMφ (solid line). Isotype controls were depicted as gray-filled histogram. (FIG. 10B) Representative histograms comparing marker expressions in HD naive circulating CMo (dashed line) and PMo (solid line). Isotype controls were depicted as gray-filled histogram.

FIG. 11A-E depict in vitro differentiation of CMos into PMos. Purified HD CD14+ CMo were cultured under various conditions (TC-treated dishes, cell-repellent dishes, cell-repellent dishes with shear flow, and endothelial cell-coated dishes with shear flow) in X-VIVO™ 15 serum-free medium supplemented with CSF-1 for 3 days. (FIGS. 11A and B) Marker expressions of cultured monocyte/macrophage in four culture groups. (FIG. 11C) CMo differentiation trajectories of CMos (CD43⁻CD163⁻) into either PMos (CD43⁺CD163⁻) or MoMφs (CD43⁻CD163⁺) visualized across culture conditions. (FIGS. 11D and 11E). Temporal marker expression dynamics in monocytes cultured on cell-repellent dishes with shear flow from day 0 (D0) to day 6. Data shown as mean±SEM and are representative of three experiments performed; statistical significance assessed by two-tailed Student's t-test; * p<0.05.

FIG. 12A-D depict in vitro uptake of sickle RBCs with EC by induced-PMo. Purified CD14+ CMo from HD and SCD patients were cultured in tissue culture (TC)-treated dishes for MoMφs and in cell-repellent dishes under shear flow to induce PMos, using X-VIVO™ 15 serum-free medium supplemented with CSF-1 over three days. (FIG. 12A) Representative dot plots showed the phenotype of induced-PMos (CD43⁺CD163⁻) and MoMφs (CD43⁻CD163⁺) in above two group cultures. (FIG. 12B) Representative dot plots comparing CFSE⁺ cells from cultures of hemin-pretreated confluent HLECs with CFSE-labeled sickle RBCs and HD naive total monocyte, or HD MoMφs, or HD induced-PMos. (FIG. 12C) Representative dot plots comparing CFSE⁺ cells from cultures of hemin-pretreated confluent HLECs with CFSE-labeled sickle RBCs and SCD MoMφs or SCD induced-PMos. (FIG. 12D) Frequencies of CFSE⁺ monocyte subsets from FIG. 12B HD (n=6) and FIG. 12C patients with SCD (n=6). Data represent mean±SEM; means were compared using two-tailed Student's t-test. * p<0.05.

FIG. 13A-C depict in vivo uptake of sickle RBCs by human naive PMo. Rag2^{-/-}IL2rg^{-/-} mice were infused with Dil-labeled RBCs from either sickle mice or control mice, followed by hemin treatment. Purified HD naïve total monocytes were adoptively transferred into these mouse models. (FIG. 13A) Representative dot plots illustrated the gating strategy of human CMos and PMos from transferred HD total monocyte in mouse blood. (FIG. 13B) Representative histograms depicted the comparison of Dil⁺ human PMo and CMo from the Rag2^{-/-}IL2rg^{-/-} mice. (FIG. 13C) Frequencies of Dil⁺ human monocyte subsets in the Rag2^{-/-}IL2rg^{-/-} mice. Data are shown as mean±SEM; statistical comparisons were performed using a two-tailed Student's t-test. * p<0.05 denoted statistical significance.

FIG. 14A-C depict in vivo uptake of sickle RBCs by human induced-PMo. Rag2^{-/-}IL2rg^{-/-} mice were transfused with Dil-labeled RBCs from sickle mice or control mice, followed by hemin treatment. Cultured induced-PMos from HD and SCD patients were adoptively transferred into these mouse models. (FIG. 14A) Representative dot plots displayed the gating strategy of transferred human monocytes in mouse blood. (FIG. 14B) Representative histograms comparing Dil⁺ human monocyte from above Rag2^{-/-}IL2rg^{-/-} mice. (FIG. 14C) Frequencies of Dil⁺ human monocyte subsets in above Rag2^{-/-}IL2rg^{-/-} mice. Data represent mean±SEM; means were compared using two-tailed Student's t-test. * p<0.05.

### DETAILED DESCRIPTION

Sickle cell disease (SCD) is an inherited hemoglobinopathy characterized by painful vaso-occlusive events (VOE or vaso-occlusive crises (VOC)) and chronic hemolysis. The mononuclear phagocyte system plays a crucial role in SCD pathophysiology. In the present disclosure the terms vaso-occlusive events (VOE) and vaso-occlusive crises (VOC) are used interchangeably.

Patrolling monocytes (PMo) are a subset of white blood cells (WBCs) which play a key role in vascular inflammation, removing damaged cells from the vasculature, thereby ensuring improved blood flow. In mouse models of Alzheimer's disease and atherosclerosis, which have inflamed vessel walls, increasing the number of patrolling monocyte improves vascular health. A protective role for PMo has been described in wound healing, in resolution of inflammation, and in clearing tumor metastases.

In SCD, sickle red blood cells (RBCs), which are inherently sticky, attach to the vessel walls and block blood flow. These VOE are characteristics of the disease and are associated with painful crises and organ damage. Patrolling monocytes can phagocytose sickle red cells that are attached to the vessel wall. Furthermore, in a mouse model of sickle cell disease, increasing the number of PMo improves vaso-occlusion (reduces VOE), while the depletion of PMo increases vaso-occlusion, exacerbating organ damage.

Increasing patrolling monocyte numbers can lead to improved vascular homeostasis in sickle cell disease, thereby alleviating painful events. Currently, no protocols (or therapeutics) exist to expand PMo to harness their therapeutic effects. In a mouse co-culture system consisting of monocytes with feeder cells, PMo can be expanded. A “monocyte culture cocktail” has been identified to replace feeder cells, which will lead to development of novel therapeutic modalities.

Blood monocytes can be subdivided based on both phenotype and function into three subsets: classical (CMo), intermediate, and patrolling (also sometimes referred to as non-classical) monocytes. Under homeostatic conditions, 90-99% of circulating CMo will transmigrate across the vascular endothelium into tissues and differentiate into macrophages or dendritic cells.

In contrast, only about 1-10% of circulating CMo differentiate into blood PMo. Once differentiated, these monocytes have a longer circulating half-life than CMo under normal conditions, and rarely cross the endothelium conditions or differentiate into macrophages. It is well established that human monocytes in culture differentiate into macrophages within 1-2 weeks. Mouse bone marrow-derived monocytes will also differentiate into macrophages (identified as loss of Ly6C and induction of MHC class II, F4/80, CD64) in the presence of CSF-1 within 5-7 days.

In the presence of IFN-γ, purified human CMo express markers associated with differentiated monocytes, although the differentiation was only to the intermediate monocyte subset stage. To date, no culture conditions exist for differentiation of monocytes (human or mouse) into PMo.

Hemolysis induces colony-stimulating factor 1 (CSF-1) production, in part by endothelial cells (ECs) through Nrf2, leading to differentiation of CMo into PMo. However, hemolysis through type I interferon (IFN-I) mediates upregulation of CCL-2, also induces transmigration and differentiation of CMo into monocyte-derived macrophages. Blockade of CMo transmigration leads to increase in circulating PMo, confirming that differentiation into tissue macrophages occurs at the expense of CMo to PMo differentiation in SCD. There is a positive association between plasma CSF-1/CCL-2 ratios and blood PMo numbers in SCD patient samples, supporting the clinical relevance of these two opposing pathways in monocyte differentiation. In a murine model of SCD, treatment with a combination of CSF-1 and anti-P-selectin antibody, a transmigration blockade, increases PMo numbers and diminishes stasis more effectively than single-agent treatments. The experiments disclosed herein provides new insights into the regulation of monocyte fates by the balance between two hemolysis pathways, Nrf-2-CSF-1 and IFN-I-CCL-2 and suggests that manipulation of the CSF-1/CCL-2 ratio could offer a novel approach for diagnostics and therapeutics in SCD.

Previous data has shown that some purified mouse bone-marrow CMo, when cultured (RPMI in 10% fetal bovine serum) with mouse brain-derived endothelial cell line (bEND.3) feeders for one day, trans-migrate across the endothelium. These trans-migrated cells have the phenotype of a differentiated macrophage, identified as loss of Ly6C and induction of MHC class II. After 2 days in culture, the cells which have not migrated, and which remain above the endothelium, display the phenotype of PMo (identified as loss of Ly6C without induction of MHC-II, F4/80 or CD64).

When bEND.3 feeder cells are pre-treated with anti-P-selectin before monocytes are added, an almost doubling of the number of patrolling monocyte-like cells are detected. Pretreatment of bEND.3 with anti-vCAM-1, or anti-ICAM-1 also increases the patrolling monocyte numbers. Treatment with anti-CD11b (myeloid-specific) at the start of the co-cultures also almost doubles the patrolling monocyte numbers.

Deficiency of patrolling monocyte numbers or function is associated with vascular dysfunction. Patrolling monocytes can also be used as a cell therapy product to target vascular inflammation, to facilitate wound healing, and to clear tumor metastasis.

Thus, disclosed herein is a novel culture system to expand PMo ex vivo, enabling the use of the cells as a cell therapy product for patients who have deficiency of PMo to correct their vascular dysfunction or to bolster their numbers to target vascular inflammatory states, for wound healing, and to clear tumor metastasis.

Patrolling monocytes expanded using the disclosed culture conditions, from monocytes isolated from patient's blood, are infused into the patients similar to other classical autologous cell therapy products. The PMo can also be expanded from allogeneic blood sources and provided as an allogenic product. In some embodiments, a library or bank of allogeneic PMo are produced that can be HLA-matched to recipients. In some embodiments, the PMo are HLA-matched to the recipient. In some embodiments, the PMo are not HLA-matched to the recipient.

Disclosed herein are methods of differentiating monocytes into PMo ex vivo, the method comprising: obtaining CMo from a subject; culturing the CMo in the presence of colony stimulating factor-1 (CSF-1); wherein as a result of the culture conditions, the CMo differentiate into PMo. In some embodiments, the CMo are purified from the bone marrow monocytes or derived from induced pluripotent stem cells (iPSC). In some embodiments, the PMo have the phenotype of CD43⁺CD163⁻. In some embodiments, the feeder cells are endothelial cells, mesenchymal stromal cells, or fibroblasts. In some embodiments, the endothelial cells are brain-derived endothelial cells. In some embodiments, the endothelial cells are murine brain-derived endothelial cells (bEND.3). In some embodiments, the feeder cells are pre-treated with one or more of an antibody specific for P-selectin, an antibody specific for vCAM-1, an antibody specific for CD11b, or an antibody to ICAM-1 prior to culturing with the CMo. In some embodiments, the culture takes place in a transwell culture system. In some embodiments, the culture takes place in the absence of feeder cells.

In some embodiments, the culture takes place on a cell-repellent surface. Surfaces used in cell culture are generally designed for cell adherence. As used herein, the term “cell-repellent surface” refers to a culture surface that prevents cells attachment to the surface. Non-limiting examples of cell-repellent surfaces include the CELLSTAR® surface manufactured by Greiner Bio-One and the BIOFLOAT™ surface manufactured by faCellitate,

In some embodiments, the culture conditions include shear stress. In their natural environment in vivo, many cell types are surrounded by moving fluids. The liquid flow around cells causes shear stress, a mechanical force that influences the cell morphology and behavior. In vitro cell culture with liquid flow, and shear stress, simulates that natural movement of fluids around cells and produces a more physiological, in vivo-like environment. Non-limited examples of shear stress used in the disclosed culture methods include culturing cells on a rocker platform or on an orbital shaker or on cell culture system under flow (ibidi® Pump System Quad).

Also disclosed are methods of expanding the number of PMo in a subject, the method comprising: administering to the subject CSF-1 and an antibody specific for P-selectin; wherein the method induces the differentiation of CMo to PMo in the subject and thus increases the number of PMo in the subject.

Also disclosed are methods of increasing the number of PMo in a subject comprising increasing the levels of CSF-1 and/or decreasing the levels of CCl2 in the subject.

Also disclosed are methods of reducing VOE and/or pain in a subject with sickle-cell disease (SCD) comprising increasing CSF-1 and/or decreasing CCl2 in the subject.

Also disclosed are methods of treating SCD in a subject in need thereof comprising: administering to the subject CSF-1 and an antibody specific for P-selectin; wherein the method increases the number of PMo in the subject, thereby treating the SCD. In some embodiments, the combination of CSF-1 and an antibody specific for P-selectin treats a vaso-occlusive event in the subject better than the antibody specific for P-selectin alone. In some embodiments, the antibody specific for P-selectin is crizanlizumab. In some embodiments, the PMo are autologous to the subject. In some embodiments, the PMo are allogenic to the subject.

Also disclosed are methods of treating SCD in a subject in need thereof, comprising administering to the subject the PMo produced by the methods disclosed herein. In some embodiments, the PMo are autologous to the subject. In some embodiments, the PMo are allogenic to the subject.

Also disclosed are methods of treating a vascular inflammatory condition in a subject in need thereof, comprising administering to the subject the PMo produced by the methods disclosed herein. In some embodiments, the PMo are autologous to the subject. In some embodiments, the PMo are allogenic to the subject.

Also disclosed are method of promoting wound healing in a subject in need thereof, comprising administering to the subject the PMo produced by the methods disclosed herein. In some embodiments, the PMo are autologous to the subject. In some embodiments, the PMo are allogenic to the subject.

Also disclosed are methods of reducing tumor metastasis, comprising administering to the subject the PMo produced by the methods disclosed herein. In some embodiments, the PMo are autologous to the subject. In some embodiments, the PMo are allogenic to the subject.

Also disclosed are methods of monitoring the efficacy of a pharmaceutical agent in the treatment of SCD or a VOE in SCD in a subject in need thereof, the method comprising: a) measuring the ratio of CSF-1 to CCL2 (CSF-1/CCL2) in the blood of the subject, wherein the CSF-1/CCL2 ratio is indicative of expected efficacy of the pharmaceutical agent in treating SCD; or (b) measuring the number of blood PMo in the subject, wherein a high level of blood PMo is indicative of expected efficacy of the pharmaceutical agent in treating SCD. In some embodiments, a high ratio of CSF-1/CCL2 is predictive, or indicative, of efficacy of the antibody in treatment of SCD or a VOE in SCD. In some embodiments, a low ratio of CSF-1/CCL2 is predictive, or indicative, of non-efficacy of the antibody in treatment of SCD or a VOE in SCD. In some embodiments, a high number of PMo is predictive, or indicative, of efficacy of the antibody in treatment of SCD or a VOE in SCD. In some embodiments, a low number of PMo is predictive, or indicative, of non-efficacy of the antibody in treatment of SCD or a VOE in SCD. In some embodiments, the ratio of CSF-1/CCL2 or the number of PMo are measured before initiation of antibody therapy. In some embodiments, the ratio of CSF-1/CCL2 or the number of PMo are measured after initiation of antibody therapy. In some embodiments, the ratio of CSF-1/CCL2 or the number of PMo are measured more than 6 months after the initiation of antibody therapy. In some embodiments, the determination of the ratio of CSF-1/CCL2 or the number of PMo causes a change in the therapy for the subject. In some embodiments, efficacy is a reduction in the number of VOE in the subject.

Also disclosed are methods of determining the risk of VOE in SCD in a subject in need thereof, the method comprising: (a) measuring the number of PMo in the blood of the subject, wherein a low level of PMo is a risk factor for a VOE; or (b) measuring the ratio of CSF-1 to CCL2 (CSF-1/CCL2) in the blood of the subject, wherein a lower CSF-1/CCL2 ratio is a risk factor for a VOE.

Also disclosed are methods of predicting the efficacy of a pharmaceutical agent in the treatment of SCD or a VOE in SCD in a subject in need thereof, the method comprising: (a) measuring the ratio of CSF-1 to CCL2 (CSF-1/CCL2) in the blood of the subject, wherein the CSF-1/CCL2 ratio is indicative of expected efficacy of the pharmaceutical agent in treating SCD; or (b) measuring the number of PMo in the blood of the subject, wherein a high level of PMo is indicative of expected efficacy of the pharmaceutical agent in treating SCD. In some embodiments, a high ratio of CSF-1/CCL2 is predictive of efficacy of the pharmaceutical agent in treatment of SCD or a VOE in SCD. In some embodiments, a low ratio of CSF-1/CCL2 is predictive of non-efficacy of the pharmaceutical agent in treatment of SCD or a VOE in SCD. In some embodiments, a high number of PMo is predictive of efficacy of the antibody in treatment of SCD or a VOE in SCD. In some embodiments, a low number of PMo is predictive of non-efficacy of the antibody in treatment of SCD or a VOE in SCD.

As used herein, the high number of PMo comprises a number of PMo more than about 25 PMo/microliter of blood, or more than about 28 monocytes/μl, about 30 monocytes/μl, about 32 monocytes/μl, about 35 monocytes/μl, about 37 monocytes/μl, about 40 monocytes/μl, or about 45 monocytes/μl of blood. As used herein, the low number of PMo comprises a number of PMo less that about 25 PMo/microliter of blood, or less than about 23 monocytes/μl, about 20 monocytes/μl, about 18 monocytes/μl, about 15 monocytes/μl, about 12 monocytes/μl, or about 10 monocytes/μl of blood.

In some embodiments, the pharmaceutical agent is L-glutamine, voxelotor, hydroxyurea, or an antibody specific for P-selectin. In some embodiments, the pharmaceutical agent is an antibody specific for P-selectin. In some embodiments, the antibody specific for P-selectin is crizanlizumab or inclacumab. In some embodiments, the antibody specific for P-selectin is an antibody disclosed in WO2012/088265 which is incorporated by reference herein for all it discloses regarding P-selectin specific antibodies.

### EXAMPLES

### Example 1. Hemolysis Dictates Monocyte Differentiation Through Two Distinct Pathways: Implications for Controlling Vaso-Occlusion in Sickle Cell Disease

Human samples. The study was approved by the Institutional Review Boards of the New York Blood Center, Montefiore Medical Center, and University of Illinois Hospital and Clinics. Blood samples were obtained after informed consent from patients with SCD, all homozygous for hemoglobin S. A cohort of patients undergo a chronic transfusion therapy (every 3-4 weeks for minimum of two years using leuko-depleted units, phenotype matched for the C, E, and K red cell antigens), immediately pre-transfusion. Race-matched control samples were obtained from de-identified healthy volunteer donors (HD) of the New York Blood Center. All blood specimens underwent processing within 18 hours of collection. Patient clinical characteristics are detailed in Table 1. A cohort of patients at high risk for acute vaso-occlusive crisis (VOC; the terms VOC and VOE (vaso-occlusive event) are used interchangeably herein) were on the therapy of crizanlizumab (n=6, median age: years, range; % (n=) females). Acute VOCs are defined as sudden episodes characterized by widespread pain manifesting in the upper or lower extremities, back, chest, and abdomen, and are associated with SCD.

TABLE 1
SCD patient information

| Parameters | Patients with SCD | |
|---|---|---|
| Number | n = 30 | |
| Age (years), median (min, max) | 22 | (14, 61) |
| Female | 20 | (67%) |
| Transfusion | 17 | (57%) |
| Hydroxyurea treatment | 16 | (53%) |
| Hemoglobin S (%), median (min, max) | 72.4 | (25.6, 91.1) |
| Splenectomy | 2 | (7%) |
| WBC (×10³/μl), median (min, max) | 9.55 | (4.1, 17.3) |
| Neutrophil (×10³/μl), median (min, max) | 5.28 | (2, 12.4) |
| Lymphocyte (×10³/μl), median (min, max) | 2.7355 | (1.32, 6.109) |
| Monocyte (×10³/μl), median (min, max) | 0.60 | (0.17, 1.06) |
| Hemoglobin (g/dl), median (min, max) | 9.05 | (6.8, 13.3) |
| Reticulocyte (×10³/μl), median (min, max) | 293.8 | (137.2, 675.2) |

| | |
|---|---|
| Platelet (×10³/μl), median (min, max) | 364 81, 748) |

Mice. All mice experiments were approved by the New York Blood Center's Animal Care and Use Committee. HbSS-Townes sickle mice (homozygous for β^{S}) and HbAA-Townes control mice (homozygous for BA) were obtained by breeding HbAS-Townes mice (013071, The Jackson Laboratory). C57BL/6J wild-type (WT) mice (000664), Tlr4^{-/-} mice (029015), Ifnar1^{-/-} mice (028288), and FVB mice (001800) 0) were acquired from Jackson Laboratory. Vav1-cre⁺Nrf2floxp^{+/+} (Vav1-cre⁺Nrf2^{+/+}) mice deficient in Nrf2 in the hematopoietic lineage and endothelial cells were obtained from Dr. Larry Luchsinger by crossing Vav1-Cre mice (008610) with Nrf2floxp mice (025433). All mice were bred in house, fed a standard rodent chow diet, and housed in micro-isolator cages in a special pathogen-free facility.

Plasma preparation and cell isolation. Whole blood samples were analyzed for complete blood counts and leukocyte differentials utilizing the Advia® 120 Hematology Analyzer (Siemens Healthcare Diagnostics). For the isolation of peripheral blood mononuclear cells (PBMCs), human blood samples underwent centrifugation at 282×g for 10 min. Subsequently, supernatants were removed for plasma preparation and white cell pellets were subjected to density gradient centrifugation using Ficoll (GE Healthcare) to isolate peripheral blood mononuclear cells (PBMCs).

Mouse white blood cells were prepared from whole blood by lysing red blood cells (RBCs) with RBC Lysis Buffer (Thermo Scientific). Plasma was obtained from the whole blood supernatant through centrifugation at 500×g for 10 min. Mouse liver single cell suspensions were generated using a liver dissociation kit (Miltenyi Biotec) in combination with the gentleMACS™ Octo Dissociator with Heaters (Miltenyi Biotec) according to the manufacturer's guidelines. RBCs within the organ single-cell suspensions were lysed using RBC Lysis Buffer.

Antibodies, Flow Cytometry and ELISA. Fluorescently labeled anti-human antibodies against CD14 (M5E2), CD16 (3G8), HLA-DR (G46-6), and CD45 (HI30) were purchased from BD Biosciences. Human Fc blocker (human IgG) were purchased from Miltenyi Biotec. LIVE/DEAD Fixable Violet Dead Cell Stain (ViViD®) and DAPI were obtained from Thermo Scientific. Fluorescently labeled mAbs anti-mouse CD45 (30-F11), CD11b (M1/70), CD64 (X54-5/7.1), F4/80 (BM8), Ly-6C (HK1.4), Ly-6G (1A8), CD11c (N418), MHC-II (M5/114.15.2), CD24 (M1/69), Tim-4 (RMT4-54), and anti-mouse CD16/32 (93, as Fc blocker) were purchased from Biolegend. Unconjugated anti-mouse CSF-1 polyclonal antibody (goat) were purchased from R&D Systems and conjugated using the Zenon™ labeling Kit (Thermo Scientific) before use.

For the assessment of marker expression in human and mouse specimens via flow cytometry, single cells were prepared and first stained with the dead cell marker ViViD®, and after two washes, were fixed/permeabilized with intracellular fixation and permeabilization buffer set (Thermo Scientific) following manufacture's instructions. Cells were incubated with Fc blocker and then stained with various fluorescent antibodies in different staining configurations. Data acquisition was performed on an LSR Fortessa flow cytometer (BD Biosciences) and the resulting data were analyzed utilizing the FlowJo™ software (Tree Star).

To quantify the plasma concentrations of CSF-1 in human and mouse samples, ELISA kits from R&D Systems and Biolegend were employed, respectively. For the evaluation of CCL2 plasma levels in both human and mouse specimens, the Cytometric Bead Array (CBA) kit (BD Biosciences) was used, according to the provided manufacturer's protocol.

Hemin and RBC lysate preparation. Hemin was purchased from Frontier Scientific and was dissolved in 0.2M NaOH, followed by neutralization to a pH of 7.2 using HCl. For the preparation of RBC lysates, purified RBC pellets from WT mice were lysed in water (10-fold volume) at room temperature for 30 min. Cellular debris was then pelleted by centrifugation at 13,000×g for 15 min. Supernatants were retained and supplemented with 10×PBS ( 1/10 volume). Total heme levels in the supernatants were tested using QuantiChrom™ heme assay kit (BioAssay Systems).

In vivo treatment. To investigate the induction of CSF-1 in vivo, mice were injected intravenously (i.v.) with freshly prepared hemin (8.8-35 μmol/kg body weight), RBC lysate (17.5 μmol heme/kg body weight), or muramyl dipeptide (MDP, InvivoGen, 1 mg/kg body weight/day), while PBS (200 μl/20 g body weight) served as a control. In some experiments, hemin was combined with hemopexin at a 1:1 ratio. For the administration of exogenous CSF-1, mice were injected subcutaneous (s.c., into the loose skin over the neck) with recombinant human CSF-1 (0.5 mg/kg body weight/day, PeproTech) or PBS as a control for four consecutive days. To block endogenous CSF-1 activity, mice were injected intraperitoneally (i.p.) with a blocking antibody against CSF-1 (5A1) or isotype control (1 mg/kg body weight, Bio X Cell). For blockade of CMo migration in vivo, mice were treated with Ultra-LEAF™ (Low Endotoxin, Azide-Free) blocking antibodies against CD62p (RB40.34, BD), CD106 (M/K-2.7, Bio X Cell), or isotype control (Bio X Cell) (5 mg/kg body weight, i.v.). Mice were sacrificed at various time points post-treatment, and blood samples and liver tissues were collected for subsequent analysis.

In vitro co-culture of CMo and EC within transwell system. CMo were purified from WT murine bone marrow using negative selection approach with a cocktail of biotin-labeled antibodies against CD19 (6D5), CD3 (145-2C11), TER-119 (TER-119), Ly-6G (1A8), NK1.1 (PK136), CD170 (M1305A02), CD11c (N418), and MHC-II (M5/114.15.2) (all from Biolegend), followed by anti-biotin microbeads (Miltenyi) according to the manufacturer's instructions. The in vitro co-culture experiments were conducted using 6.5 mm transwell inserts with 3.0 μm pore polycarbonate membrane and CellAdhere™ Collagen I-Coated 24-Well Flat-Bottom Plates (Stem Cell). Purified CMo were added onto the insert pre-cultured with confluent mouse endothelial cell line bEnd.3 [BEND3] (ATCC). The co-culture was evaluated after a 48-hour incubation period. To block CMo migration, Ultra-LEAF™ blocking antibodies against CD62p (RB40.34, BD), CD106 (M/K-2.7, Bio X Cell), CD62E (9A9, Bio X Cell), CD11b (M1/70, Biolegend) CD54 (YN1/1.7.4, Biolegend) or isotype control (Bio X Cell) were added 30 min prior to initiating the co-culture at a concentration of 10 ng/ml.

Histological analysis. Histological and immunohistochemical assessments were conducted by the Laboratory of Comparative Pathology (LCP) at Weill Cornell Medicine/MSKCC. Tissue samples were fixed in formalin, embedded in paraffin, and sectioned at 5 μm thicknesses. The sections were stained with hematoxylin and eosin (H&E) (Sigma-Aldrich), or terminal deoxynucleotidyl transferase-mediated dUTP nick-end labeling (TUNEL) according to standard procedures and examined using a Leica DM 2000 microscope.

Statistical analysis. The data in each experiment were analyzed separately and displayed as individual data points in figures. Data are presented as mean values±SEM. Statistical analyses were performed using GraphPad Prism (GraphPad software). To determine the statistical significance of the differences between experimental groups, a two-tailed Student's t-test was employed. A p-value of less than 0.05 was considered statistically significant.

### Results

### Hemolysis Induce CSF-1 Production in SCD

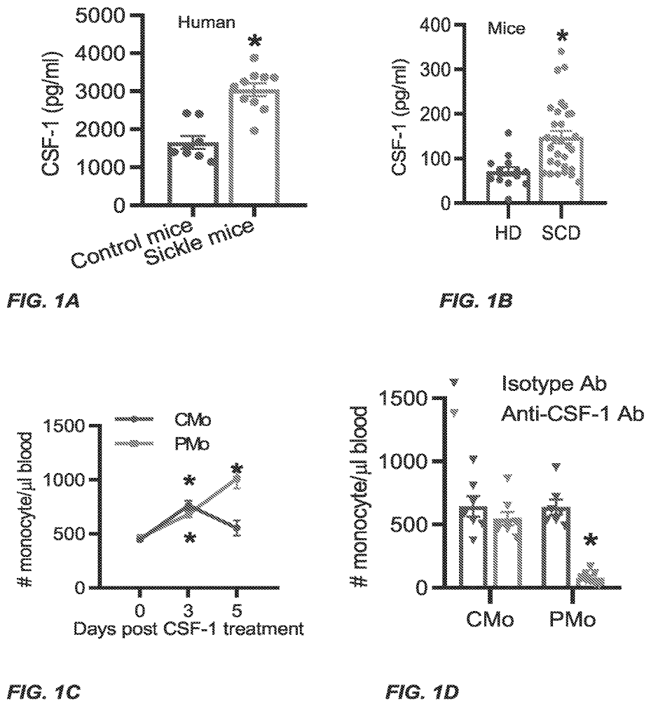

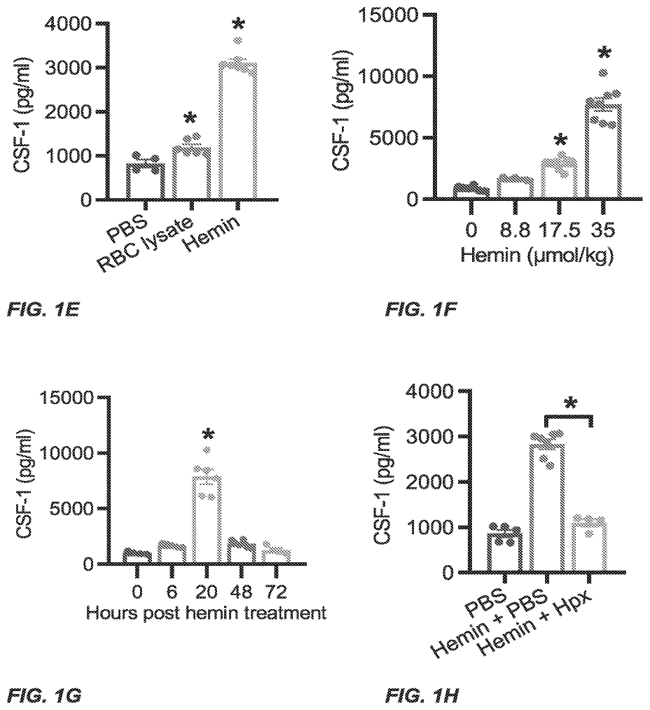

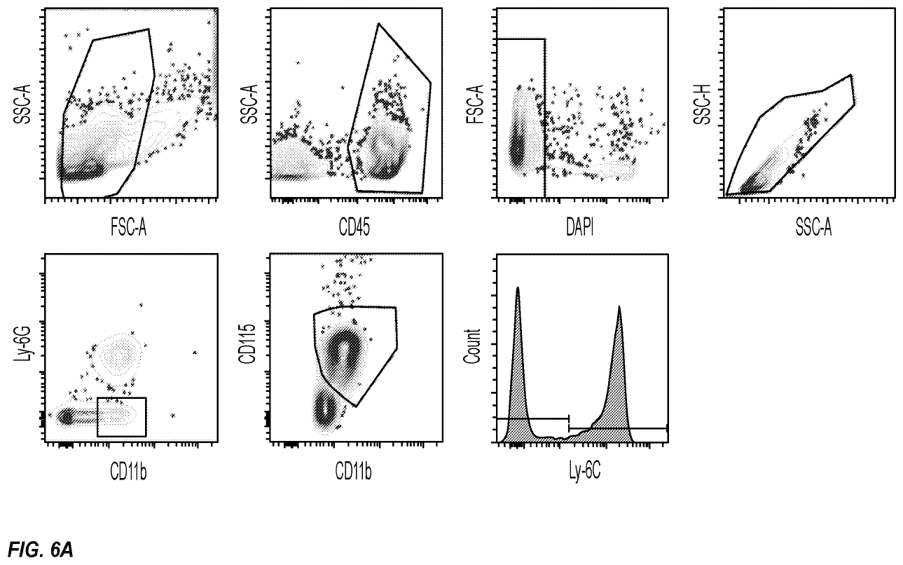

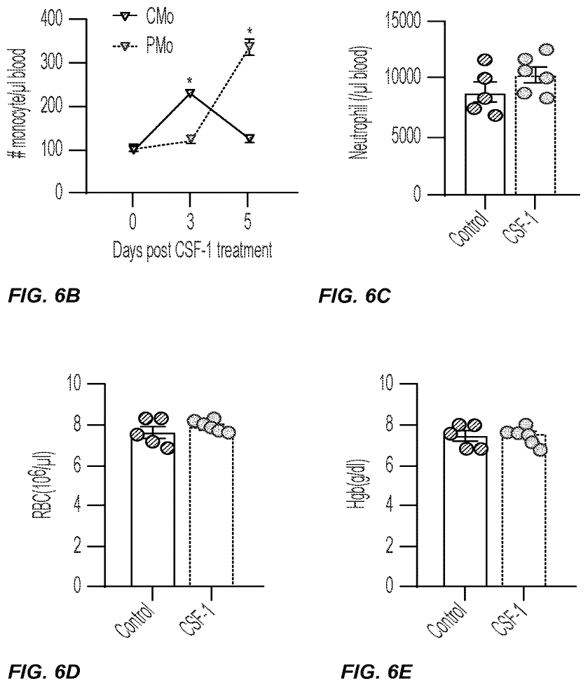

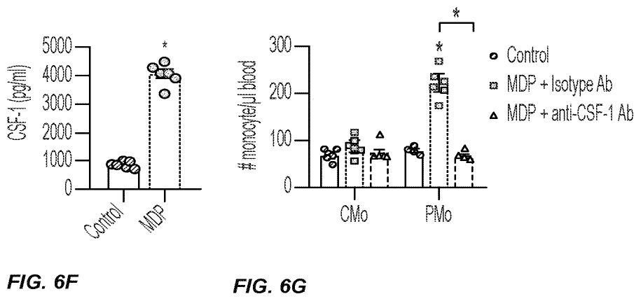

Monocytosis is a characteristic feature of SCD. Since colony stimulating factor (CSF-1) is a crucial growth factor for monocyte survival and differentiation, elevated circulating CSF-1 is seen in SCD patients compared to healthy donors (HD) (FIG. 1A, two-fold, p<0.05, Table 1), and in the Townes mouse model of SCD (sickle mice) relative to control mice (FIG. 1B, 1.7-fold, p=0.002). Injection of recombinant CSF-1 led to a two-fold increase in the numbers of blood CMo on day 3 and PMo on day 5 in sickle mice (FIG. 1C, p<0.05 compared to PBS treatment, see gating strategies in FIG. 6A) and as previously demonstrated in primates including humans, a similar change in wild-type (WT) mice (FIG. 6B, p<0.05) (Munn et al. Blood 88:1215-1224, 1996; Schmid et al. Cytometry 22:103-110, 1995). In contrast, treatment with a blocking anti-CSF-1 antibody in sickle mice resulted in a significant 8-fold reduction in circulating PMo numbers and a trend toward a decrease in circulating CMo (FIG. 1D, compared to isotype antibody control) in WT mice. Moreover, treatment with MDP, a PMo inducer, led to increased plasma CSF-1 levels in WT mice while pre-treatment with anti-CSF-1 antibodies attenuated PMo expansion induced by MDP (FIGS. 6C and D, p<0.05). To investigate whether hemolysis, a hallmark of SCD, can upregulate CSF-1 production, we induced acute hemolysis in WT mice using RBC breakdown products. Following injection of RBC lysate or hemin, plasma CSF-1 levels increased by 50% and 4-fold, respectively (FIG. 1E, p<0.05, compared to PBS control). We observed a dose dependent increase in CSF-1 induction with escalating concentrations of hemin (FIG. 1H, p<0.05). Plasma CSF-1 was first detectable at 6 hr post-hemin injection, peaked at 20 hr, and returned to baseline levels at 72 hr (FIG. 1G, p<0.05). Administration of hemin with heme scavenger hemopexin in wild-type (WT) mice completely abrogated the increase in plasma CSF-1 (FIG. 1H, p<0.05), substantiating the role of hemin in CSF-1 induction. Collectively, these findings demonstrate that intravascular hemolysis triggers CSF-1 production and CSF-1 is a key regulator of PMo numbers in SCD.

### Hemin-Induced CSF-1 is Produced Partly by ECs Through Nrf2 Pathway

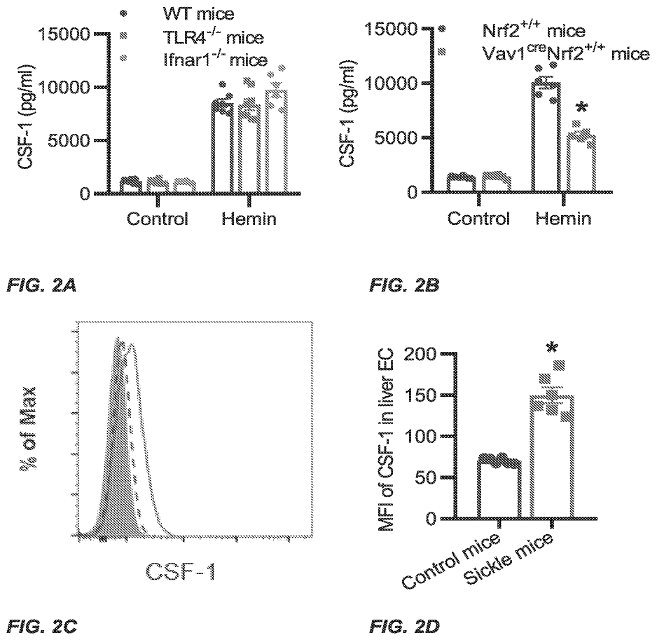

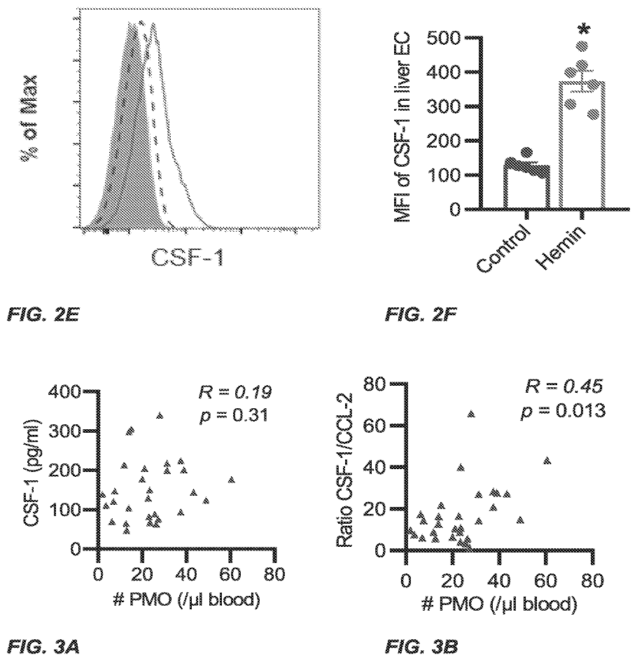

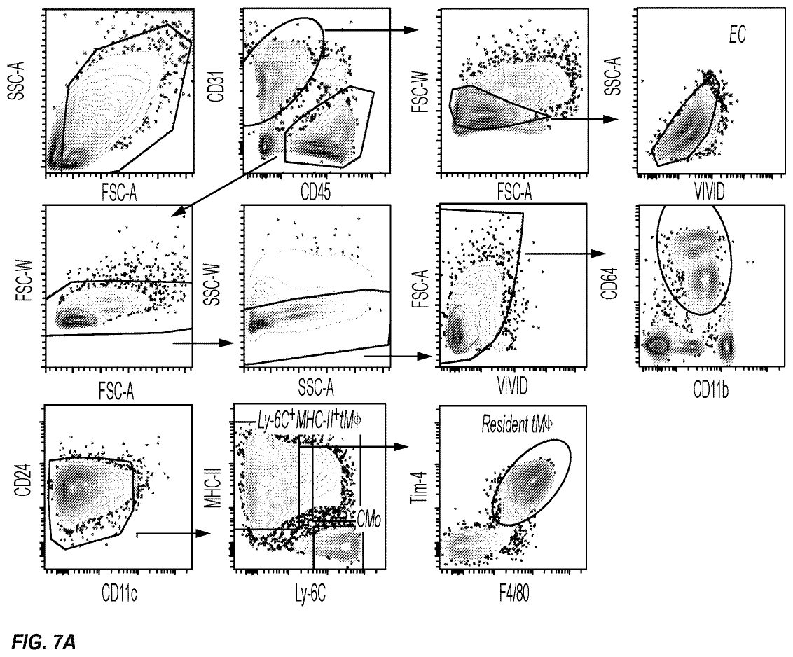

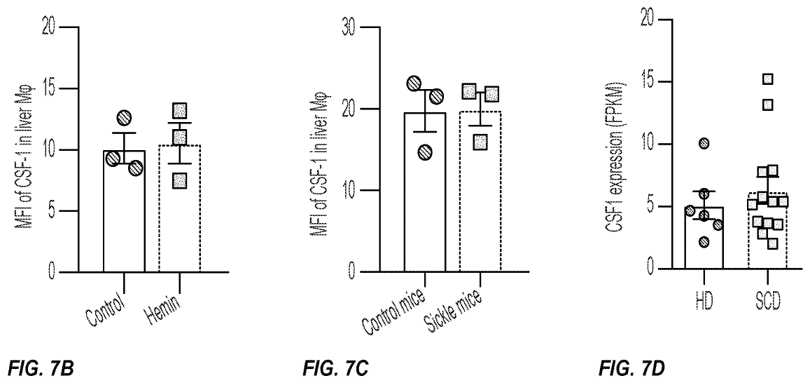

Since cell-free heme can activate TLR4 and IFN-I pathways, we assessed the effect of hemin on the upregulation of plasma CSF-1 in TLR4^{-/-} and ifnar^{-/-} mice. Interestingly, comparable CSF-1 levels were observed following hemin treatment in both gene knockout mouse strains as in WT mice (FIG. 2A). Heme is also known to activate the Nrf-2 transcription factor, which upregulates a broad array of antioxidant enzymes that protect against hemolysis. To evaluate the involvement of the heme-Nrf2 pathway in CSF-1 induction, we employed Vav1-cre⁺Nrf2floxp^{+/+} (Vav1^{cre}Nrf2^{+/+}) mice, which lack Nrf2 in the hematopoietic lineage and endothelial cells. In these mice, we observed a 50% reduction in hemin-induced plasma CSF-1 levels compared to Nrf2^{+/+} control mice (FIG. 2B, p<0.05). To identify the cellular origin of CSF-1 in SCD, we analyzed CSF-1 protein levels using flow cytometry. Concentrating on the hepatic tissue, which serves as the primary heme detoxification organ, we observed elevated CSF-1 signals in liver ECs of sickle mice compared to control mice (FIGS. 2C and D, 2-fold, p<0.05; see gating strategies in FIG. 7A), and in hemin-treated WT mice relative to PBS-treated mice (FIGS. 2E and F, 2.7-fold, p<0.05). Although low levels of CSF-1 were detected in macrophages, no significant differences were found between sickle and control mice or between hemin-treated and PBS-treated WT mice (FIGS. 7B and 7C, see gating strategies in FIG. 7A). Furthermore, CSF-1 signals were essentially undetectable in all circulating monocyte subsets in the blood of sickle mice, in line with the minimal CSF-1 transcripts (5 FPKM) observed in human SCD peripheral blood monocytes (FIG. 7D) (Liu et al. Blood 138:1162-1171, 2021). Collectively, these findings demonstrate that ECs, but not monocytes/macrophages, produce CSF-1 in SCD in response to hemolysis through the Nrf2 pathway and independent of TLR4 or IFNa1R signaling.

CMo to PMo Differentiation in Response to Hemolysis Correlates with Circulating CSF-1/CCL-2 Ratios

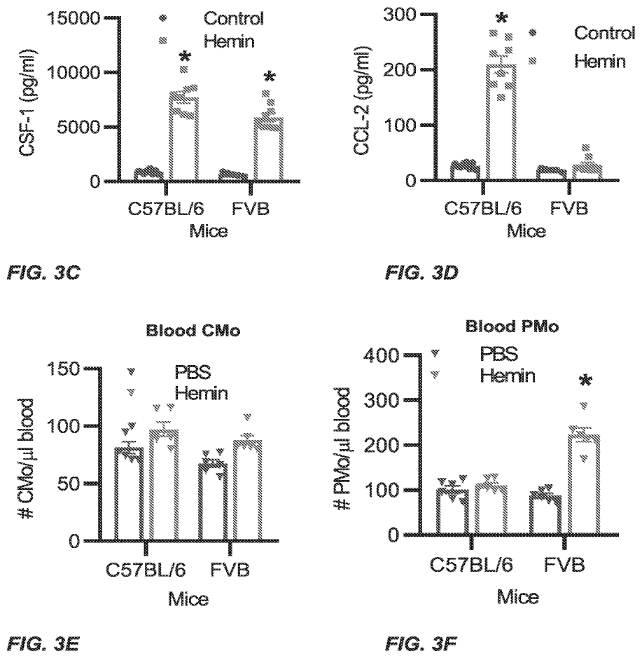

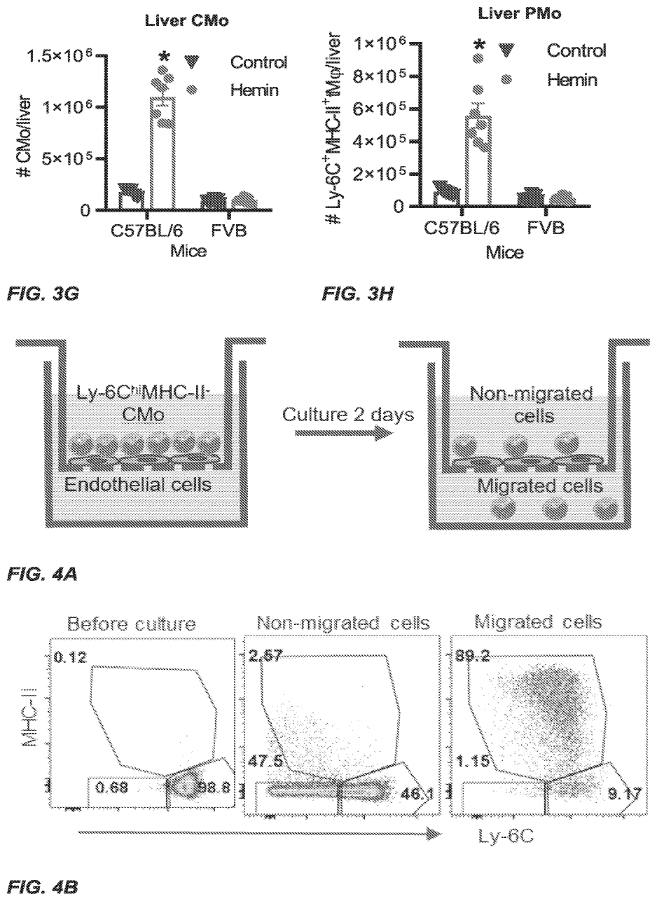

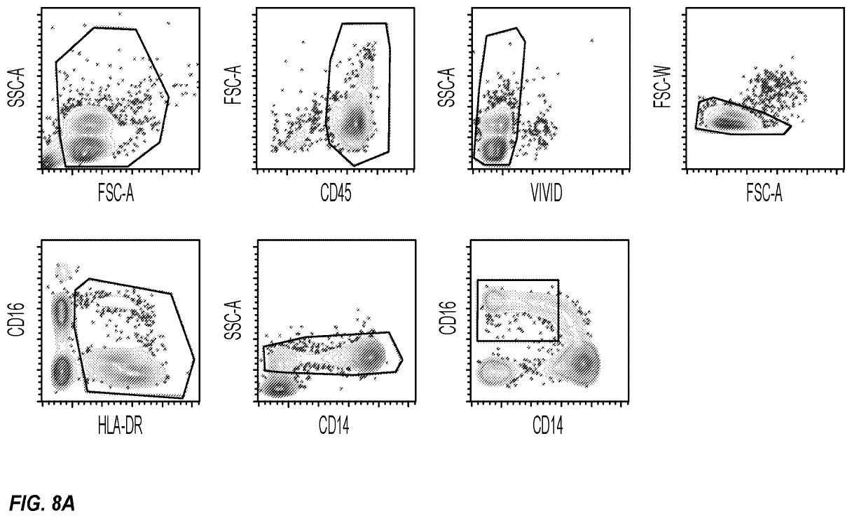

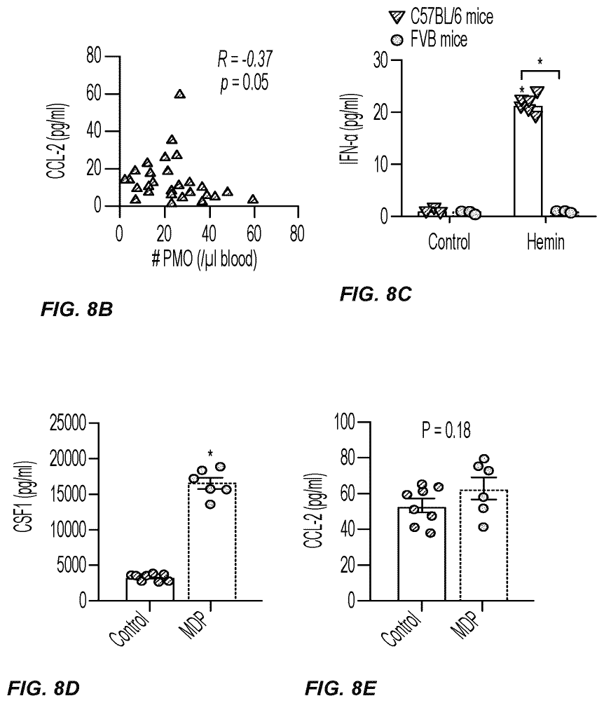

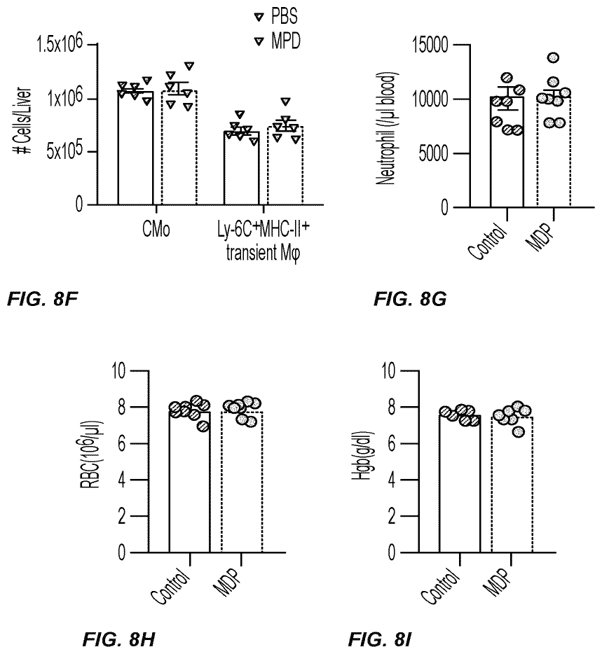

To establish the relationship between CSF-1 and PMo numbers in SCD, we conducted correlation analyses, but surprisingly, no association was found (FIG. 3A, R=0.13, P=0.49), suggesting a role for other factors that may impact CSF-1 control of PMo numbers in SCD. We have previously demonstrated that hemolysis results in upregulation of IFN-α-induced CMo chemokine (CCL-2) and subsequent CMo migration and differentiation into tissue monocyte-derived macrophages (Liu et al. Blood 138:1162-1171, 2021). Although PMo numbers did not directly correlate with CCL-2 levels (FIG. 8A), we found a significant positive correlation in SCD patients between PMo numbers (see gating strategies in FIG. 8B) and the CSF-1/CCL-2 ratio (FIG. 3B, R=0.47, P=0.009). These data suggest that the balance between CSF-1 and CCL-2 regulates blood PMo numbers in SCD. Interestingly, we observed a differential response to hemin in the induction of CSF-1 and CCL-2 in two mouse strains, WT C57BL/6 mice and FVB mice. Both mouse strains showed a 9-fold upregulation of circulating CSF-1 levels (FIG. 3C, one-day post hemin treatment p<0.05), but only C57BL/6 mice, but not FVB mice, showed increases in IFN-α (FIG. 8C) and CCL-2 (FIG. 3D, approximately 8-fold, p<0.05). Importantly, an increase in circulating PMo numbers was only detected in FVB mice (high CSF-1/CCL-2 ratio), but not in C57/BL/6 (FIGS. 3E and F, day 3 post-injection p<0.05). In contrast, elevated liver CMo and Ly-6C⁺MHC-II⁺transient macrophages were exclusively found in C57BL/6 mice (low CSF-1/CCl-2 ratio; FIGS. 3G and 3H, see gating strategies in FIG. 7A). Additionally, we found that MDP treatment resulted in a significant upregulation of CSF-1 but not CCL-2 in sickle mice, accompanied by an increase in circulating PMo numbers (Liu et al. Blood 134:579-590, 2019) but not liver CMo and monocyte-derived macrophages (Ly-6C⁺MHC-II⁺transient macrophage) (FIG. 8D-F). Blood neutrophil numbers, RBC numbers, and hemoglobin levels in sickle mice did not show a change after MDP treatment (FIG. 3G-I). In response to hemolysis, a higher upregulation of CSF-1 along with low induction of CCL-2 promotes CMo to PMo differentiation in circulation, thus increasing blood PMo numbers as observed in FVB mice. Conversely, if CCL-2 is also adequately induced as observed in C57BL/6 mice, the outcome is tissue migration and differentiation of CMo into monocyte-derived macrophages instead of differentiation to PMo in blood.

### Blockade of CMo Migration Increases Circulating PMo but Reduces Liver Monocyte-Derived Macrophages

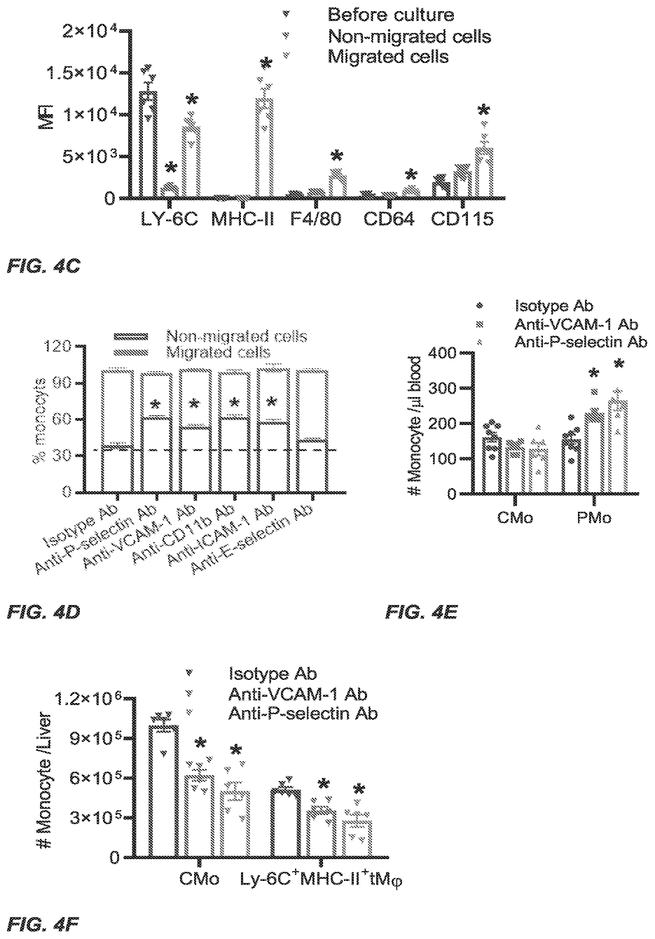

To establish that transmigration and differentiation of CMo into macrophages occurs at the expense of CMo to PMo differentiation, we employed an in vitro transwell culture model wherein purified mouse CMo, identified as Ly-6C⁺MHC-II⁻ cells, were placed in the upper chamber of the transwell which were pre-seeded with ECs to simulate CMo trans-endothelial migration. Monocytes that migrated across ECs into the bottom well exhibited MHC-II expression (FIG. 4A-C), implying their predisposition towards Ly-6C⁺MHC-II⁺transient macrophage differentiation. Interestingly, monocytes which did not migrate but remained in the top well did not display elevated MHC-II expression (FIG. 4A-C). Instead, they exhibited a characteristic PMo-like phenotype, marked by decreased Ly-6C levels and lower expression of macrophage markers such as F4/80, CD64, and CD115 compared to migrated cells (FIG. 4C, p<0.05). These data suggest that non-migrated CMo on ECs can differentiate into PMo. Adhesion molecules play a critical role in CMo trans-endothelial migration. To examine whether inhibiting CMo transmigration across ECs through adhesion molecule blockade mediated increased PMo differentiation, we pretreated ECs in the transwell culture system with blocking antibodies targeting key adhesion molecules including P-selectin, VCAM-1, CD11b, ICAM-1, E-selectin, or isotype control. With every antibody except anti-E-selectin antibody, we observed an increased frequency of non-migrated cells (mostly PMo-like monocytes) and a concomitant decrease in migrated cells (mostly Ly-6C⁺MHC-II⁺transient macrophages) (FIG. 4D, p<0.05), suggesting that impeding CMo trans-endothelial migration may promote PMo differentiation.

We next tested whether these blocking antibodies had an effect on monocyte numbers in an in vivo hemolysis model. In hemin-injected WT mice pre-treated with blocking antibodies against VCAM-1 or P-selectin, compared to isotype control antibody, we found a two-fold increase in blood PMo numbers along with a 50% decrease in liver CMo numbers and 20% reduction in Ly-6C⁺MHC-II⁺transient macrophages (FIGS. 4E and F, p<0.05). Blocking antibodies against E-selectin demonstrated no substantial efficacy in modulating monocyte migration and differentiation processes in this in vivo model (FIGS. 8A and B).

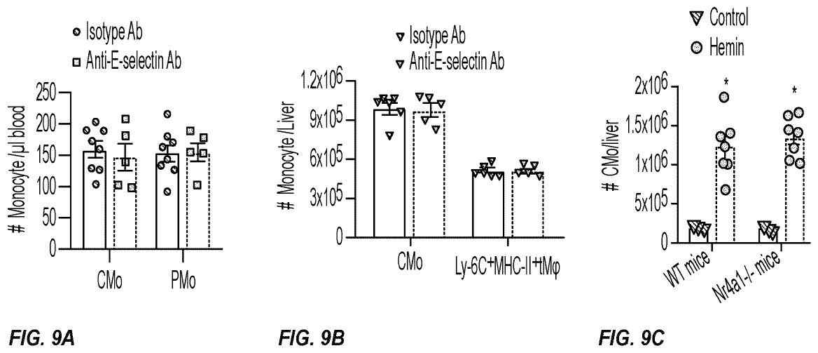

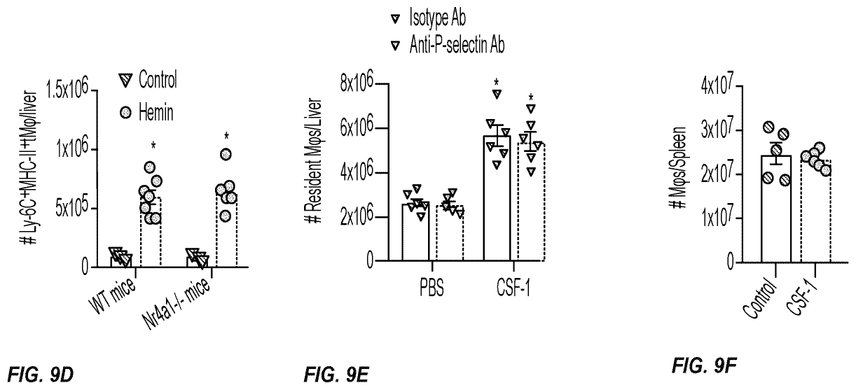

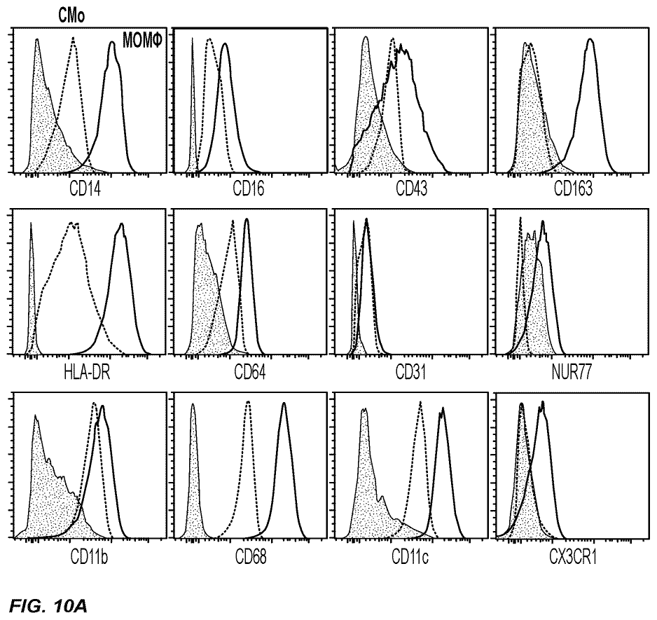

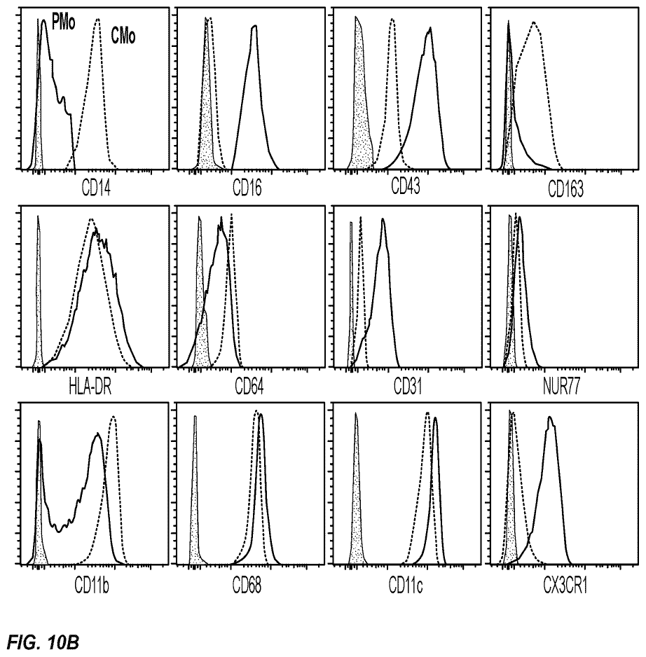

In non-hemolytic conditions, CMo can differentiate into PMo first and then differentiate into macrophages. To determine whether this differentiation pathway is also relevant within a hemolysis context, we administered hemin to both WT and Nr4a1 knockout mice (characterized by a lack of PMo). However, the induced liver CMo and Ly-6C⁺MHC-II⁺transient macrophage following hemolysis was comparable in both WT and Nr4a1 knockout cohorts (FIGS. 9C and D), suggesting that the pathway for hemolysis-induced liver monocyte-derived macrophage differentiation is independent of PMo. Collectively, our findings demonstrate that obstructing CMo trans-endothelial hepatic migration, which is induced by hemolysis, can increase circulating PMo numbers, while reducing tissue monocyte-derived macrophages.

### CSF-1 and Anti-P-Selecting Antibodies Induce Circulating PMo and Reduce VOC in Sickle Mice

We previously established that modulating PMo numbers in sickle mice can alter red cell stasis. (Liu et al 2019). We hypothesized that CSF-1-induced PMo might prevent VOC, and that a combination of CSF-1 and anti-P-selectin antibody might even further enhance PMo numbers to thus improve VOC.

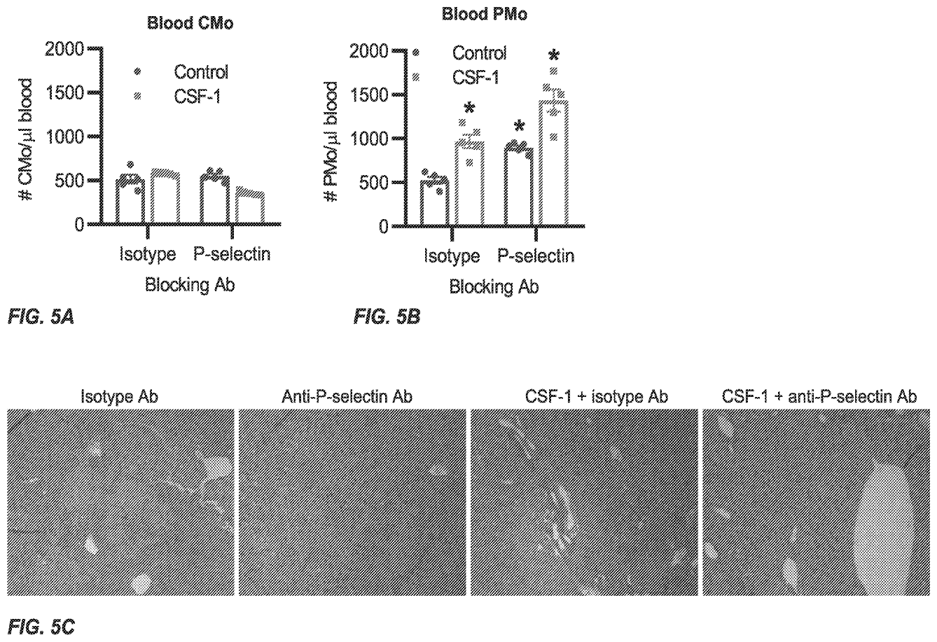

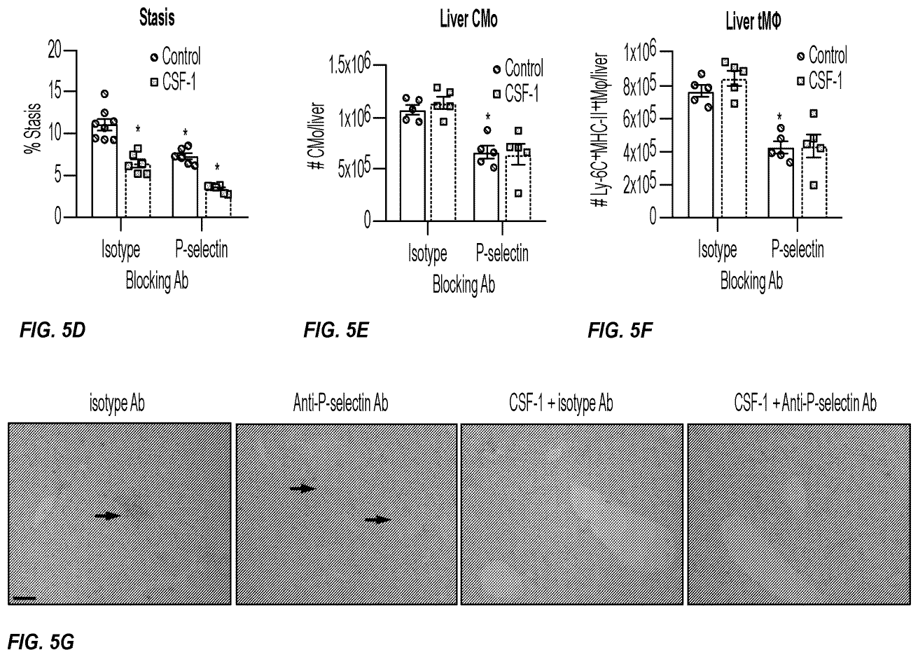

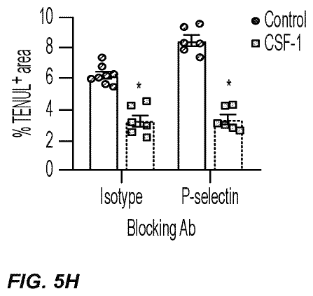

Sickle mice were administered anti-P-selectin, or isotype control antibodies, or a combination of CSF-1 and anti-P-selectin or isotype control antibodies. Consistent with the data in FIG. 1, CSF-1 combined with the isotype antibody led to a rise in circulating PMo numbers compared to the isotype antibody alone, whereas CSF-1 paired with anti-P-selectin antibody resulted in even higher circulating PMo levels compared to each alone (FIGS. 5A and 5B, p<0.05). Histological (H&E) analysis of liver vasculature revealed diminished vascular stasis (by half) in sickle mice treated with CSF1 and isotype antibody, or anti-P-selectin antibody alone compared to isotype antibody-treated mice, which was further enhanced in CSF-1 and anti-P-selectin antibody-treated mice (FIGS. 5C and 5D, p<0.05). Treatment with CSF-1 combined with the isotype antibody did not alter liver CMo and Ly-6C⁺MHC-II⁺transient macrophage numbers in sickle mice; however, treatment with anti-P-selectin antibodies (which blocks CMo migration) with or without CSF-1 resulted in decreases in these numbers (FIGS. 5E and 5F, p<0.05). Macrophages play a crucial role in protecting against liver damage in SCD. Unlike Ly-6C⁺MHC-II⁺transient macrophage numbers, we observed an increase in liver resident macrophage (F4/80^{hi}Tim-4⁺, see gating strategies in FIG. 7A) numbers following CSF-1 administration (FIG. 9E, p<0.05), as in WT mice, but not in spleen macrophage numbers (FIG. 9F). However, anti-P-selectin antibody had no effect on resident macrophage numbers (FIG. 9E). TUNEL staining of liver tissue revealed diminished liver injury in sickle mice treated with CSF-1 and isotype or anti-P-selectin antibody compared to either antibody without CSF-1 (FIGS. 5G and 5H). In mice treated with anti-P-selectin antibody alone, liver injury was slightly increased (FIGS. 5G and 5H), which is in line with recent data in P-selectin deficient mice. Altogether, these data demonstate that, compared to either treatment alone, combination therapy with CSF-1 and anti-P-selectin further increases PMo numbers and is more efficacious in prevention of VOC. Furthermore, CSF-1 drives expansion of macrophages that prevent liver damage.

### Discussion

In this study, we demonstrate that hemolysis regulates monocyte fate through two distinct pathways in SCD. Using the Townes SCD mouse model, we found that heme-induced production of CSF-1, primarily by tissue ECs via Nrf2, promotes the differentiation of CMo into PMo. On the other hand, heme-induced CCL-2 through IFN-I drives mouse blood CMo transmigration into tissues and their differentiation into monocyte-derived macrophages. We also found that the relative ratio of plasma CSF-1 and CCL-2 levels, both elevated in SCD, directly correlates with blood PMo numbers in SCD patients, suggesting that the balance between CSF-1 and CCL-2 pathways dictates circulating monocyte fates under hemolytic conditions. Based on our mouse data that CMo to monocyte-derived macrophage differentiation occurs at the expense of blood PMo differentiation, we established an in vitro mouse EC culture system for expansion of PMo from CMo by inhibiting transmigration. Importantly, combination therapy with CSF-1 and anti-P-selectin blocks monocyte transmigration into tissues, further bolstering PMo numbers and conferring better protection against stasis in SCD mice than either treatment alone.

A key finding of our study is that PMo numbers in SCD are determined by the CSF-1/CCL-2 ratio. It is well-established that CMo can be expanded by CSF-1, migrate into tissue induced by CCL-2, and subsequently differentiate into macrophages or dendritic cells under both steady-state and pro-inflammatory conditions. Intravenously injected MDP, a peptidoglycan motif common to all bacteria, has been shown to induce PMo production in various mouse models including SCD. Our data reveals that PMo expansion by intravenous MDP drives higher upregulation of blood CSF-1 compared to CCL-2. This is in contrast to a previous report showing that in a bacterially induced Crohn's disease model, MDP which was produced in tissues, led to the upregulation of CCL-2 (Kim Y G et al. Immunity 34:769-80, 2011). The response to intravenous exposure to MDP may be different from locally produced MDP, although in that report, neither CSF-1 levels nor the effect on circulating PMo numbers were measured. Our analysis centered on the roles of CSF-1 and CCL-2 on monocyte differentiation, since these two factors are upregulated in hemolytic conditions (hemin treatment) and SCD. Our observations particularly highlighted an increase in plasma CSF-1 levels was mainly attributed to ECs, although a role for mesenchymal stromal cells/fibroblasts which are also a major source of CSF-1, cannot be excluded. Our findings have focused on the role of cell-free heme but do not preclude the potential involvement of other damage-associated molecular patterns (DAMPs) released during intravascular hemolysis in the regulation of CSF-1 production in SCD. Other growth factors and chemokines may also contribute, such as CSF-2, which plays a crucial role in at least mouse monocyte development. Interestingly, circulating levels of CSF-2 are reported to be elevated in SCD patients. However, administration of anti-CSF-2 blocking antibodies did not affect the numbers of blood PMo in sickle mice (446±39/μl blood vs 432±48/μl blood with isotype antibody treatment), indicating a CSF-2-independent regulation of blood PMo number in our mouse model.

Additionally, CX3CL1 and its receptor CX3CR1 are crucial for PMo migration, although it remains controversial whether blood PMo numbers are reduced in Cx3CR1 knockout mice. Other chemokines, such as CCL7, CCL8, and CCL12, influence monocyte migration but are not considered as critical as CCL-2 and exhibit less monocyte specificity, as they also attract various other WBCs. CCL2 plasma levels and PMo numbers/frequency are also altered in endotoxemia, myocardial infarction, and malaria. Thus, the relevance of CSF-1/CCL2 ratio to blood PMo numbers may be extended to other disease states. We observed a modest reduction in total monocyte numbers in patients on hydroxyurea compared to those not receiving the treatment, which is consistent with previous reports. It has been shown that patients with SCD on hydroxyurea had higher frequency of PMo when compared to those receiving transfusions. Hydroxyurea treatment leads to a reduction in circulating classical monocyte numbers but due to a compensatory mechanism to maintain total circulating monocyte numbers, PMo lifespan is extended and therefore PMo frequency is increased in treated patients, although PMo numbers are not.

It is generally accepted that CMo do not differentiate into PMo when cultured in vitro. Numerous studies, including those presented herein, have shown that in the presence of ECs, CMo transmigrate and differentiate into macrophage-like cells. Under such culture conditions, non-migrated cells adopt PMo-like phenotypes. These discoveries pave the way for devising novel protocols that utilize ECs as feeder cells for ex vivo PMo expansion, as a first step toward developing a cell therapy product. EC-mediated regulation of CMo to PMo differentiation could occur through direct EC-CMo interactions. For example, it has been shown that the interaction of EC expressed Delta-like 1 (DLL1) with monocyte Notch2 receptor promotes CMo to PMo differentiation in vitro. However, the same group has also shown that DLL1 can induce CMo to macrophage differentiation in vitro, raising the possibility that DLL1-Notch2 signaling might not be specific for PMo differentiation. Alternatively, ECs may act as a barrier that prevents exposure to the unique extravascular tissue environment that otherwise supports CMo differentiation into monocyte-derived macrophages or even dendritic cells. Thus, CMo would differentiate into PMo in the bloodstream by a default pathway if they do not cross the ECs into tissues. However, it should be noted that hematopoietic organs such as bone marrow and spleen are an exception since they harbor specialized niche microenvironments that support CMo to PMo differentiation.

An unexpected finding of the data presented herein is that co-administration of CSF-1 in conjunction with P-selectin blockade synergistically augmented PMo numbers and diminished stasis in sickle mice more effectively than utilizing either anti-P-selectin antibody or CSF-1 alone. To a lesser extent, P-selectin blockade alone also increased blood PMo numbers and reduced stasis in sickle mice consistent with P-selectin knockout sickle mice exhibiting increased circulating monocytes and decreased liver myeloid cells, but the monocyte/macrophage populations in the latter mouse model have yet to be characterized. In this study, we found that CSF-1 plus anti-P-selectin antibody treated mice displayed reduced liver injury, possibly due to increased resident tissue macrophages in response to CSF-1, which is known to promote tissue macrophage proliferation. This is in contrast to heightened liver tissue injury observed with anti-P-selectin antibody treatment alone or P-selectin knockout sickle mice. To date, no hepatic abnormalities have been reported in patients with SCD on crizanlizumab. These data raise the compelling rationale for concomitant use of CSF-1 and anti-P-selectin antibody in SCD since it could serve dual functions: mitigating vaso-occlusion by elevating circulating PMo numbers while simultaneously bolstering tissue resident macrophage populations to protect against organ damage. Thus, we propose that the combined administration of crizanlizumab, a humanized anti-P-selectin antibody licensed as a treatment option for VOC, and CSF-1 may yield even greater efficacy in SCD management. Mechanistically, we also propose that the ability of crizanlizumab to increase PMo numbers may serve as an additional mechanism of action for safeguarding against VOC.

In conclusion, our study utilizing a mouse model of SCD has pinpointed hemolysis as a key factor in SCD promoting CSF-1 production, leading to PMo expansion if CCL2, which promotes the monocyte-derived macrophage pathway, is relatively suppressed. Novel or existing therapies that increase the CSF-1/CCL2 ratio will be more efficacious in reducing pain. A heightened PMo number achieved through the manipulation of the CSF-1/CCL2 ratio contributes to a reduced risk of VOC in SCD.

### Example 2. Human Patrolling Monocytes: Ex Vivo Expansion and Therapeutic Potential for Sickle Cell Vaso-Occlusion

Human samples. The study was approved by the Institutional Review Boards of the New York Blood Center, and Montefiore Medical Center. A cohort of patients were on chronic transfusion therapy (every 3-4 weeks for minimum of two years using leuko-depleted units, phenotype matched for the C, E, and K red cell antigens) were sampled immediately pre-transfusion. Race-matched control samples were obtained from de-identified healthy volunteer donors (HD) of the New York Blood Center. All blood specimens underwent processing within 18 hours of collection.

Mice. HbSS-Townes sickle mice (homozygous for β^{S}) and HbAA-Townes control mice (homozygous for β^{A}) were obtained by breeding HbAS-Townes mice (013071, The Jackson Laboratory). Rag2^{-/-}IL2rg^{-/-} mice (014593) were purchased from The Jackson Laboratory. Mice were fed with a standard rodent chow diet and housed in micro-isolator cages whin a special pathogen-free facility. All procedures involving mice were approved by the Animal Care and Use Committee of New York Blood Center.

Antibodies. Fluorescently labeled anti-human antibodies, including CD14 (M5E2), CD16 (3G8), HLA-DR (G46-6), CD11b (M1/70), CD68 (Y1/82A), CD163 (GHI/61), CD43 (1G10), CD64 (10.1), NUR77 (12.14), CD11c (b-ly6), and CD45 (HI30) were purchased from BD Biosciences. Anti-human CD31 (WM59) mAbs, CX3CR1 (2A9-1) mAbs, and HO-1 (HO-1-1) mAbs, Zenon labeling Kit, Annexin V, LIVE/DEAD Fixable Violet Dead Cell Stain (ViViD™), and DAPI were sourced from Thermo Scientific.

Reagents. Hemin, the oxidized form of the heme moiety of hemoglobin, was obtained from Frontier Scientific, and was dissolved in 0.2M NaOH, neutralized to pH 7.2 with HCl and adjusted to stock concentration with distilled water. Staining buffer: Phosphate-buffered saline (PBS) supplemented with 2 mM EDTA and 0.5% BSA, was used for cell staining procedures in flow cytometry.

Cell isolation and purification. Human blood samples were centrifuged at 300×g, with supernatants reserved for plasma preparation. Cell pellets then subjected to density gradient centrifugation using Ficoll (GE Healthcare) to isolate peripheral blood mononuclear cells (PBMC). Mouse white blood cells were prepared by lysing mouse whole blood samples using RBC lysis buffer.

Human total monocytes were purified using a pan-monocyte isolation kit (Miltenyi Biotec), and CD14⁺ CMo were purified using anti-human CD14 microbeads following manufacturer's instructions (purity>95%). Human and mouse RBCs were enriched from whole blood by negative selection employing anti-human CD45 microbeads and anti-mouse CD45 microbeads respectively (Miltenyi Biotec) according to the manufacturer's instructions (>95% of leukocytes depleted). Enriched RBCs were washed twice with PBS with centrifugation at 200×g for 10 min with no brake to remove platelets. In certain experiments, purified human RBCs were labeled with carboxyfluorescin diacetate succinimidyl ester (CFSE) and mouse RBCs were labeled with Vybrant™ Dil cell-labeling solution (Thermo Scientific) respectively according to the manufacturer's instructions.

Endothelial cell culture. Human liver-derived endothelial cells (HLECs) were purchased from Lonza and grown in vascular cell basal medium supplemented with endothelial cell growth kit (Lonza) at 37° C. in a humidified atmosphere of 5% CO₂. Cells were passaged and used between passages four and eight for all experiments.

In vitro monocyte differentiation. Purified human CD14⁺ classical monocyte (CMo) (2×10⁶ cells/mL) were cultured in X-VIVO™ 15 serum-free medium (Lonza) supplemented with human CSF-1 (Peprotech, 20 ng/ml). The cultures utilized various containers: first, tissue culture (TC)-treated cell culture flasks (Thermo Scientific); second, cell-repellent cell culture dishes from Greiner Bio-One (VWR); third, cell-repellent dishes with shear flow using a Scilogex SCI-0180-S LED Digital Orbital Shaker at 150 rpm (The Lab Depot); and fourth, EC-coated dishes (confluent HLECs in tissue culture (TC)-treated dishes (Thermo Scientific) with shear flow (Digital Orbital Shaker at 150 rpm). The medium was refreshed every three days. At a predetermined timepoint, cells in cell-repellent cell culture dish were collected directly, and those in TC-treated dish were harvested with cell scrapper according to the manufacturer's instructions. The collected cells underwent phenotypic and functional analyses.

Pickup of endothelial cell-attached sickle RBC by monocyte in vitro. For monocyte co-culture studies, confluent HLECs in 96-well plates were treated with hemin (20 μM) in medium for 2 hr, followed by two PBS washes to remove unbound hemin. CFSE-labeled RBC (1×10⁶/well) were added, and after 1 hr, monocytes (2×10⁵/well) were added. The co-culture was incubated overnight in RPMI-1640 medium supplemented with 100 U/ml penicillin, 100 μg/ml streptomycin, and 10% heat inactivated fetal bovine serum (Thermo Scientific). Cells were collected and stained with fluorescently labeled antibodies for flow cytometry analysis.

In vivo pickup of sickle RBC by human monocyte. Rag2^{-/-}IL2rg^{-/-} mice were intravenously injected with Dil-labeled sickle mouse RBCs (1.5×10⁹ cells/200 μl per mouse). Overnight post-RBC injection, mice received an intravenous injection of freshly prepared hemin (30 μmol/kg body weight). Four hours later, the mice underwent adoptive transfer of human monocyte (5×10⁶ cells/200 μl per mouse) and were bled 30 min afterward. Mouse white blood cells were prepared and stained with fluorescently labeled antibodies for flow cytometry analysis.

Flow cytometry. Single-cell suspensions were first labeled with the dead cell marker ViViD™ following the manufacturer's instructions. Subsequently cells were incubated with fluorescently conjugated antibodies against cell surface markers in staining buffer at 4° C. for 30 min. Following two washes with staining buffer, cells were fixed and permeabilized using intracellular fixation and permeabilization buffer set (Thermo Scientific) as per the manufacture's protocol. Cells were then incubated with fluorescently conjugated antibodies against intracellular markers at 4° C. for 30 min. After two washes with permeabilization buffer, cells were resuspended in staining buffer. The stained cells were analyzed on LSR Fortessa (BD Biosciences), and data were processed using FlowJo™ software.

Statistical analysis. Each experiment was analyzed individually, with data represented as mean±SEM. Statistical analyses were conducted using GraphPad Prism. The statistical significance of the differences between experimental groups was determined by two-tailed Student's t-test, with p-values less than 0.05 considered statistically significant.

### Results

### Markers of PMo Compared to CMo and Monocyte-Derived Macrophage.

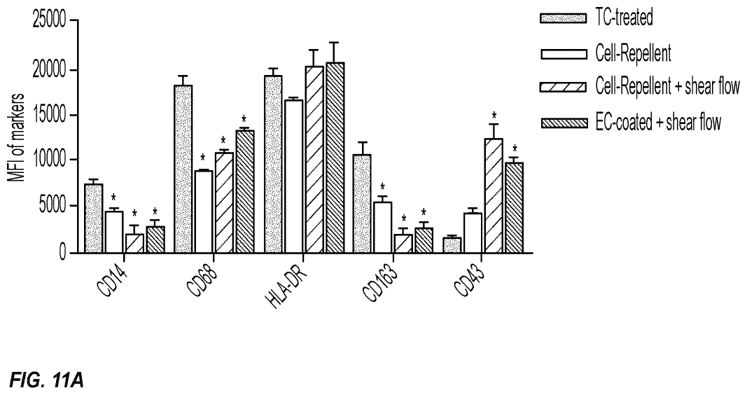

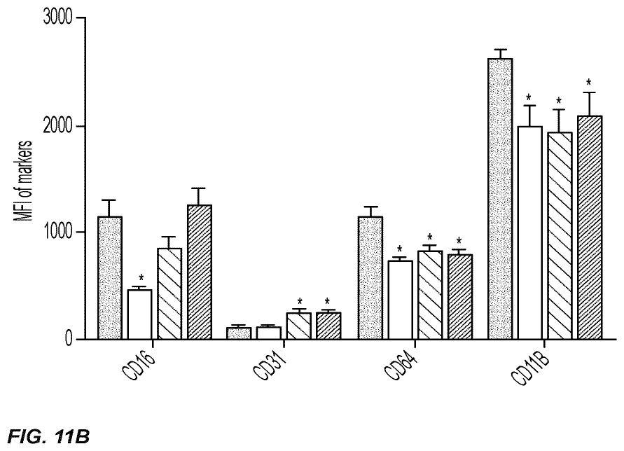

Culturing human CMos to differentiate into PMos in vitro necessitates circumventing adhesion-induced differentiation into MoMφs. A pivotal step involves establishing a methodology to differentiate between PMos and MoMφs, thus we evaluated marker expressions across naive CMos, PMos, and MoMφs. Through comparative analysis, PMos were observed to exhibit elevated levels of CD43 and CD31. Conversely, MoMφ displayed a marked increase in CD163, CD14, CD64, CD68, and CD11b markers compared to CMos. Both PMos and MoMφ showed enhanced expression of CD16, HLA-DR, CX3CR1, Nur77, and CD11c relative to CMos (FIG. 10). We choose positive selected markers (e.g. CD43) for cultured PMo and negative selected markers (e.g. CD163) to exclude MoMφ differentiation.

### Optimization of In Vitro Culture Conditions for PMo Differentiation

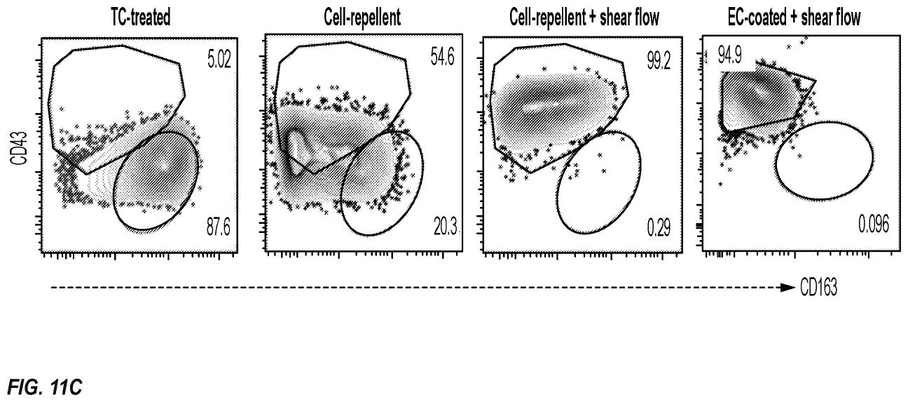

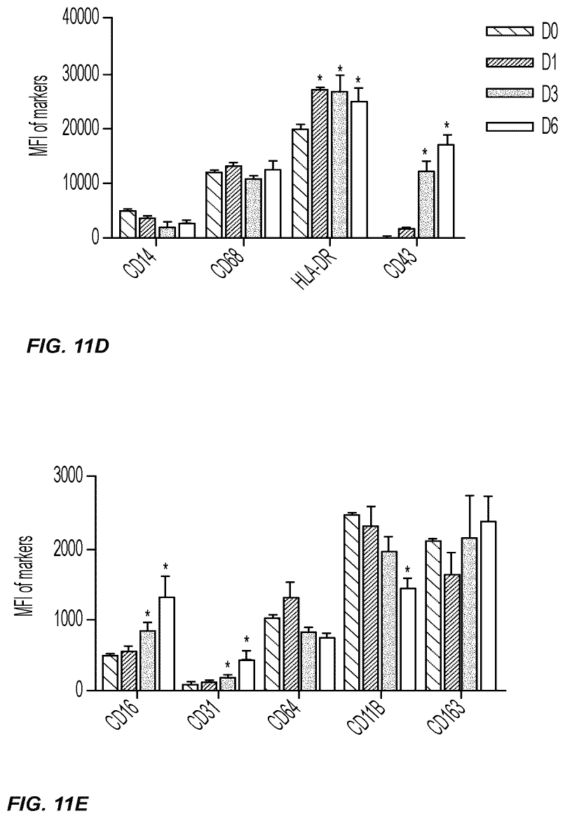

To avoid the adhesion-induced trans-differentiation of CMos into MoMφ, a series of in vitro culture environments were established: (1) tissue culture (TC)-treated dishes, (2) cell-repellent dishes, (3) cell-repellent dishes with shear flow, and (4) endothelial cell (EC)-coated dishes with shear flow. CD14⁺ CMos, isolated from healthy donors (HD), were cultured under these conditions in X-VIVO™ 15 serum-free medium supplemented with CSF-1 over a three-day period. Comparative analysis revealed that, compared with the TC-treated dishes, all other conditions favored the upregulation of CD43 and CD31, alongside a decrease in CD163, CD14, CD68, CD11b, and CD64 markers, indicating a PMo-like phenotype (FIGS. 11A and B). The most significant alterations were observed in CD43 and CD163, correlating with PMo-like and MoMφ phenotypes, respectively. A differentiation diagram was constructed to visualize the bifurcation of CMos into either PMos (CD43+CD163−) or MoMφ (CD43−CD163+) (FIG. 11C). Notably, cell-repellent dishes with shear flow and EC-coated dishes with shear flow yielded a higher propensity for CMo differentiation towards a PMo phenotype. Given the logistical advantage of not requiring EC removal, the cell-repellent dish with shear flow was identified as the optimal culture condition for generating PMos for therapeutic applications. Kinetic analysis of the differentiation process revealed significant changes in marker expression between day 3 and day 6, affirming the successful induction of a PMo-like phenotype suitable for cell therapy purposes (FIGS. 11D and E). This data conclusively demonstrates that specific in vitro culture conditions can effectively induce the differentiation of CMos into PMos, with cell-repellent dishes with shear flow emerging as the most favorable conditions.

Uptake Sickle RBCs Co-Cultured with ECs by Induced-PMos In Vitro

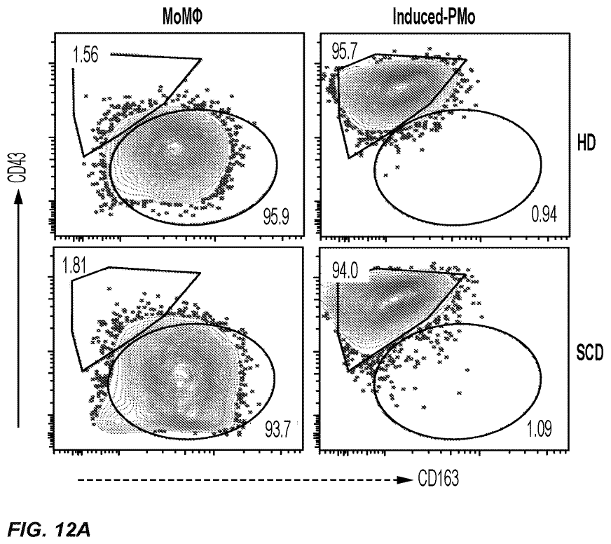

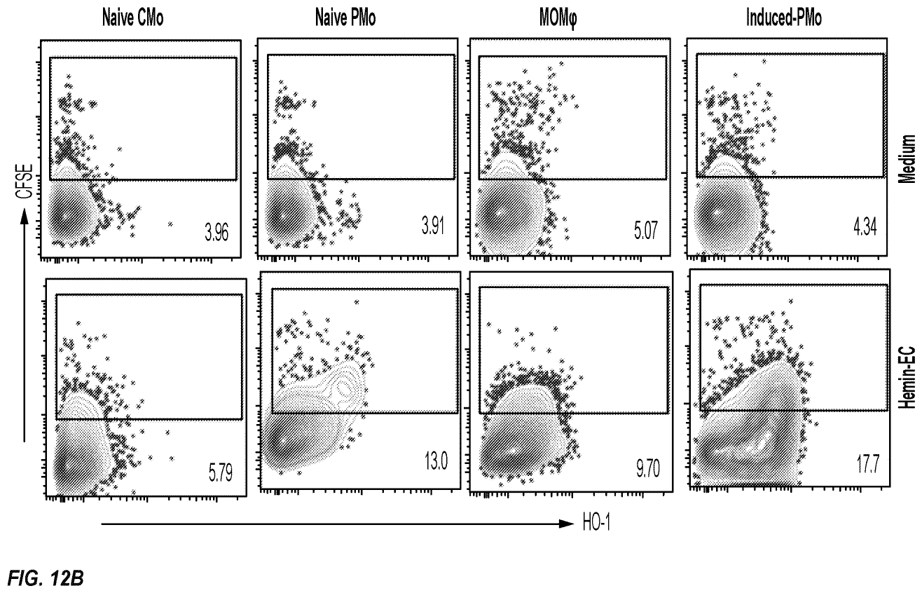

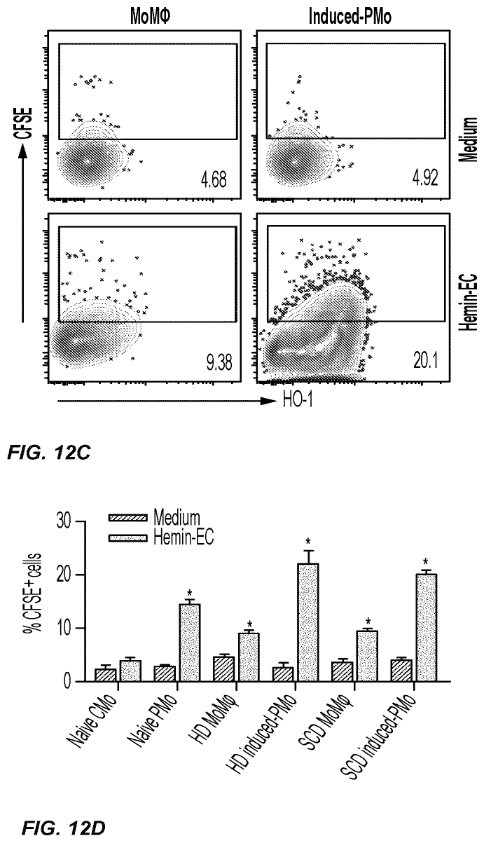

To ascertain if induced-PMos retain the functional capability of native PMos for engulfing EC-attached sickle RBCs in vitro, we initiated cultures of induced-PMos in cell-repellent dishes under shear flow conditions, alongside MoMφs cultured in TC-treated dishes, derived from both HD and SCD CMos (FIG. 12A). Subsequently, we co-cultured these cells with CFSE-labeled sickle (HbSS) RBCs alongside purified HD naive total monocytes, induced-PMos, and MoMφs, and quantified CFSE+ cell frequencies via flow cytometry, as previously presented (Liu et al. Blood 134:579, 2019). Notably, our findings revealed that, similar to naive PMos, induced-PMos demonstrated a heightened frequency of CFSE+ cells when co-cultured with sickle RBCs and ECs. Surprisingly, even MoMφs exhibited an increased frequency of CFSE⁺ cells in this co-culture, albeit to a lesser extent than both naive and induced PMos (FIG. 12B-D). We found minimal effect in the frequencies of CFSE⁺ cells from co-culture of sickle RBCs with all above monocyte or MoMφs in the presence EC (“medium” groups) (FIG. 12B-D). Altogether, these data demonstrate that sickle RBCs can be phagocytosed by induced-PMo when in contact with ECs in vitro culture system and underscore the potential of induced-PMos to mimic the phagocytic function of naïve PMo against EC-attached sickle RBCs.

### Uptake of Sickle RBCs by Induced-PMo In Vivo

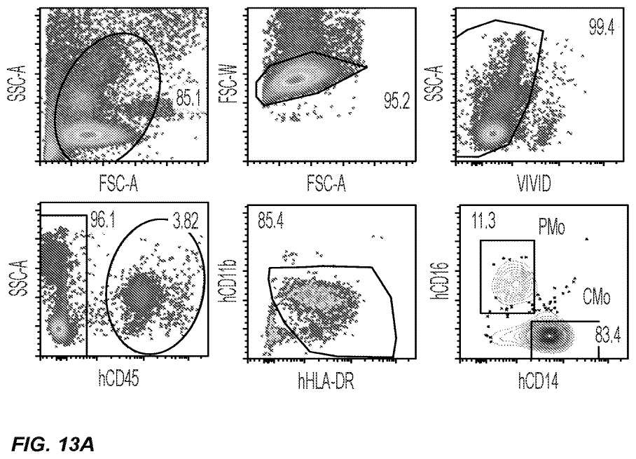

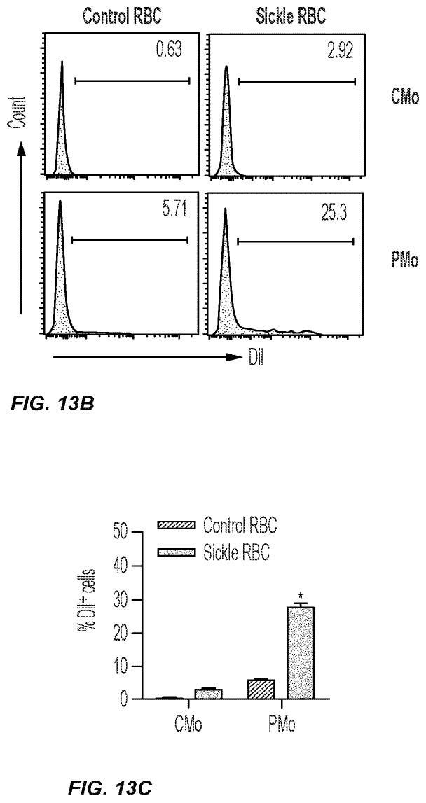

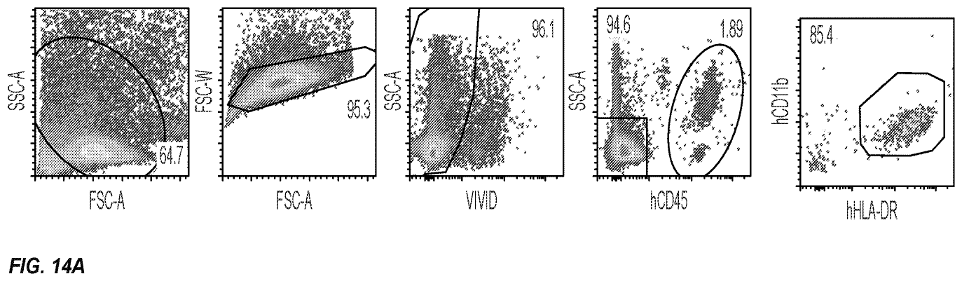

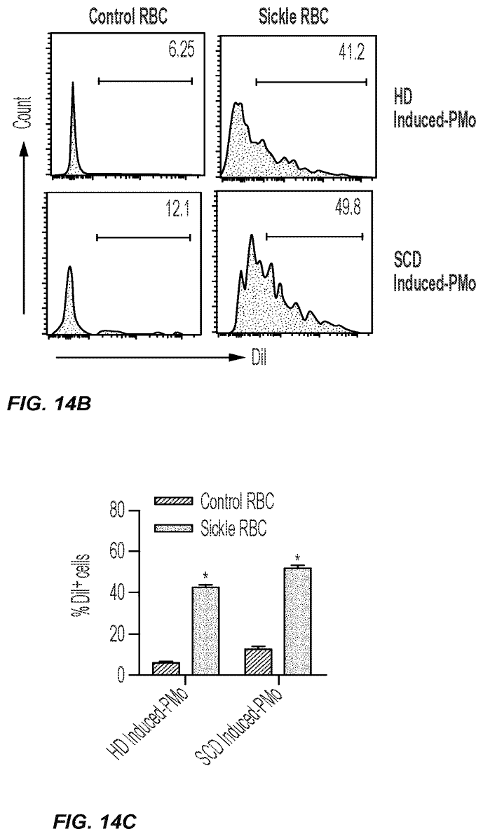

We further investigated whether human induced-PMo can uptake sickle RBCs in vivo. To test this, we utilized an in vivo SCD model with Rag2′^{-/-}IL2rg^{-/-} mice that received Dil-labeled RBCs from Townes SCD mice and followed by hemin injection to mimic SCD hemolytic conditions (Liu et al. 2019). To test whether this mouse model applies to human monocytes for sickle RBC pickup, we first adoptively transferred purified naive HD total monocytes into these mice, and clearly find these monocytes in circulation (FIG. 13A). Consistent with the in vitro data (FIG. 12), higher frequency of Dil⁺ PMo were present in mice which had been transfused with sickle RBCs, as compared to mice transfused with control RBCs (FIGS. 13B and 13C). We detected a marginal increase in Dil⁺ CMos of mice which had received sickle RBCs (FIGS. 13B and 13C). This data demonstrated that this SCD model of Rag2^{-/-}IL2rg^{-/-} mice is available for testing sickle RBC pickup by adoptive transferred human monocytes. We then adoptively transferred induced-PMos and MoMφs differentiated from HD and SCD CMos into this mice model. We found that after receiving induced-PMos from HD or SCD patients, a higher frequency of Dil⁺ induced-PMo was present in mice which had been transfused with sickle RBCs, as compared to mice transfused with control RBCs (FIG. 14). However, we did not find enough circulating human cells in mice that received MoMφs for analyzing sickle RBC pickup. Altogether these in vivo data further support the in vitro studies that induced-PMos scavenge EC-attached sickle RBCs.

### CONCLUSION

In conclusion, this investigation underscores the therapeutic promise of induced-PMos in ameliorating VOC in SCD by demonstrating their capacity for in vitro and in vivo phagocytosis of sickle RBCs.

Unless otherwise indicated, all numbers expressing quantities of ingredients, properties such as molecular weight, reaction conditions, and so forth used in the specification and claims are to be understood as being modified in all instances by the term “about.” As used herein the terms “about” and “approximately” means within 10 to 15%, preferably within 5 to 10%. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A method of differentiating monocytes into patrolling monocytes ex vivo, the method comprising:
obtaining classical monocytes;
culturing the classical monocytes in the presence of colony-stimulating factor-1 (CSF-1);
wherein as a result of the culture conditions, the classical monocytes differentiate into patrolling monocytes.

2. The method of claim or 2, wherein the patrolling monocytes have the phenotype CD43⁺CD163⁻.

3. The method of claim 1 or 2, wherein the classical monocytes are isolated from a subject.

4. The method of claim 3, wherein the classical monocytes are purified from the bone marrow monocytes.

5. The method of claim 1 or 2, wherein the classical monocytes are derived from induced pluripotent stem cells (iPSC).

6. The method of claim 5, wherein the classical monocytes are derived from HLA homozygous iPSC.

7. The method of any one of claims 1-6, the classical monocytes are cultured in the presence of feeder cells.

8. The method of claim 7, wherein the feeder cells are endothelial cells, mesenchymal stromal cells, or fibroblasts.

9. The method of claim 7 or 8, wherein the feeder cells are pre-treated with one or more of an antibody specific for P-selectin, and antibody specific for vCAM-1, an antibody specific for CD11b, or an antibody to ICAM-1 prior to culturing with the classical monocytes.

10. The method of any one of claims 7-9, wherein the culture takes place in a transwell culture system.

11. The method of any one of claims 1-6, wherein the classical monocytes are cultured in the absence of feeder cells.

12. The method of any one of claims 1-6, wherein the classical monocytes are cultured in cell repellent tissue culture dishes.

13. The method of any one of claims 1-12, wherein the classical monocytes are cultured in the presence of a shear stress.

14. A method of expanding the number of patrolling monocytes in a subject, the method comprising:
administering to the subject CSF-1 and an antibody specific for P-selectin;
wherein the method induces the differentiation of classical monocytes to patrolling monocytes in the subject and thus increases the number of patrolling monocytes in the subject.

15. A method of treating sickle cell disease (SCD) in a subject in need thereof comprising:
administering to the subject CSF-1 and an antibody specific for P-selectin;
wherein the method increases the number of patrolling monocytes in the subject, thereby treating the SCD.

16. Use of patrolling monocytes in the treatment of SCD in a subject in need thereof comprising:
administering to the subject CSF-1 and an antibody specific for P-selectin;
wherein as a result of the administration, the number of patrolling monocytes is increased in the subject, thereby treating the SCD.

17. The method of claim 15 or the use of claim 16, wherein the combination of CSF-1 and an antibody specific for P-selectin treats a vaso-occlusive event in the subject better than the antibody specific for P-selectin alone.

18. The method or use of any one of claims 15-17, wherein the antibody specific for P-selectin is crizanlizumab.

19. A method of treating SCD in a subject in need thereof, comprising administering to the subject ex vivo produced patrolling monocytes.

20. Use of ex vivo produced patrolling monocytes for the treatment of SCD in a subject in need thereof.

21. A method of treating a vascular inflammatory condition in a subject in need thereof, comprising administering to the subject ex vivo produced patrolling monocytes.

22. Use of ex vivo produced patrolling monocytes for the treatment of a vascular inflammatory condition in a subject in need thereof.

23. A method of promoting wound healing in a subject in need thereof, comprising administering to the subject ex vivo produced patrolling monocytes.

24. Use of ex vivo produced patrolling monocytes for promoting wound healing in a subject in need thereof.

25. A method of reducing tumor metastasis in a subject in need thereof, comprising administering to the subject ex vivo produced patrolling monocytes.

26. Use of ex vivo produced patrolling monocytes for reducing tumor metastasis in a subject in need thereof.

27. The method or use of any one of claims 19-26, wherein the patrolling monocytes are produced by the method of any one of claims 1-13.

28. The method or use of any one of claims 19-27, wherein the patrolling monocytes are autologous to the subject.

29. The method or use of any one of claims 19-27, wherein the patrolling monocytes are allogenic to the subject.

30. The method or use of claim 29, wherein the allogeneic patrolling monocytes are HLA matched to the subject.

31. A method of monitoring the efficacy of a pharmaceutical agent in the treatment of SCD or a vaso-occlusive events in SCD in a subject in need thereof, the method comprising:
(a) measuring the ratio of CSF-1 to CCL2 (CSF-1/CCL2) in the blood of the subject, wherein the CSF-1/CCL2 ratio is indicative of the efficacy or expected efficacy of the pharmaceutical agent in treating SCD; or
(b) measuring the number of blood patrolling monocytes in the subject, wherein a high level of blood patrolling monocytes is indicative of the efficacy or expected efficacy of the pharmaceutical agent in treating SCD.

32. The method of claim 31, wherein the pharmaceutical agent is an agent effective for treatment of SCD or effective for the prevention of vaso-occlusive events in SCD.

33. The method of either of claim 31 or 32, wherein the pharmaceutical agent is L-glutamine, voxelotor, hydroxyurea, or an antibody specific for P-selectin.

34. The method of claim 33, wherein the antibody specific for P-selectin is crizanlizumab.

35. The method of any one of claims 31-34, wherein a high ratio of CSF-1/CCL2 is predictive, or indicative, of efficacy of the antibody in treatment of SCD or a vaso-occlusive events in SCD.

36. The method of any one of claims 31-34, wherein a low ratio of CSF-1/CCL2 is predictive, or indicative, of non-efficacy of the antibody in treatment of SCD or a vaso-occlusive events in SCD.

37. The method of any one of claims 31-34, wherein a high number of patrolling monocytes is predictive, or indicative, of efficacy of the antibody in treatment of SCD or a vaso-occlusive event in SCD.

38. The method of any one of claims 31-34, wherein a low number of patrolling monocytes. is predictive, or indicative, of non-efficacy of the antibody in treatment of SCD or a vaso-occlusive event in SCD.

39. The method of any one of claims 31-38, wherein the ratio of CSF-1/CCL2 or the number of patrolling monocytes are measured before initiation of antibody therapy.

40. The method of any one of claims 31-38, wherein the ratio of CSF-1/CCL2 or the number of patrolling monocytes are measured after initiation of antibody therapy.

41. The method of claim 40, wherein the ratio of CSF-1/CCL2 or the number of patrolling monocytes are measured more than six months after the initiation of antibody therapy.

42. The method of any one of claims 31-41, wherein the determination of the ratio of CSF-1/CCL2 or the number of patrolling monocytes causes a change in the therapy for the subject.

43. The method of any one of claims 31-41, wherein efficacy is a reduction in the number of vaso-occlusive events in the subject.

44. A method of determining the risk of vaso-occlusive events in SCD in a subject in need thereof, the method comprising
(a). measuring the number of blood patrolling monocytes in the subject, wherein a low level of blood patrolling monocytes is a risk factor for a vaso-occlusive event; or
(b) measuring the ratio of CSF-1 to CCL2 (CSF-1/CCL2) in the blood of the subject, wherein a lower CSF-1/CCL2 ratio is a risk factor for a vaso-occlusive event.
